(19) United States
(12) Patent Application Publication
(10) Pub. No.: US 2022/0052370 A1
(43) Pub. Date: Feb. 17, 2022

(54) POLYMERIC ENZYME-BASED BIOFUEL CELL AND METHODS OF MAKING AND USING

(71) Applicant: King Abdullah University of Science and Technology, Thuwal (SA)

(72) Inventors: Sahika Inal, Thuwal (SA); David Ohayon, Thuwal (SA)

(21) Appl. No.: 17/296,449

(22) PCT Filed: Nov. 25, 2019
(86) PCT No.: PCT/IB2019/060051

§ 371 Date: May 24, 2021

Related U.S. Application Data
(60) Provisional application No. 62/770,934, filed on Nov. 23, 2018.

(51) Int. Cl. H01M 8/16; C12Q 1/00; H01M 4/90

(52) U.S. Cl. CPC H01M 8/16; H01M 4/9008; C12Q 1/006

(57) ABSTRACT

Enzyme-based biofuel cells including a bioanode, a biocathode, and an electrolyte solution are disclosed. The bioanode contains (a) a conductive substrate; (b) one or more n-type polymers; and (c) one or more enzymes. The biocathode contains (a) a conductive substrate; and (b) one or more p-type polymers. The electrolyte solution contains one or more metabolites capable of reacting with the enzymes and is in electrical communication with the bioanode and the biocathode. The bioanode is electrically connected to the biocathode. The biofuel generates power when the metabolites react with the enzymes to produce electrons; the electrons are directed through an electrical circuit to the biocathode where an oxidant is reduced to water. The structure of the n-type polymer allows for efficient electron transfer from the enzyme to the polymer, resulting improved biofuel cell performances. Methods of making and using the enzyme-based biofuel cells are also disclosed.

### CROSS-REFERENCE TO RELATED APPLICATION

The present application claims priority to U.S. Application No. 62/770,934, filed Nov. 23, 2018, the disclosure of which is incorporated herein by reference in its entirety.

### FIELD OF THE INVENTION

The invention is generally directed to a biofuel cell for generating energy. Specifically, the present invention is directed to a polymeric enzyme-based biofuel cell which can generate energy upon chemical transformation of metabolites.

### BACKGROUND OF THE INVENTION

Oxidation of metabolites is one of the key processes that occur in our cells breaking down chemical energy to power cellular activity. Metabolites such as glucose or lactate are endogenous substances that are readily available in biological fluids and can be continuously renewed by metabolism, which makes them ideal fuels for powering bioelectronic devices. Enzymatic biofuel cells (EFCs) that convert the energy of metabolism into electrical energy via biological pathways are one such example (Slaughter, et al., Journal of Biochips & Tissue Chips 2015, 5 (1), 1). The technological advancement of EFCs is ascribed mainly to the development of conducting materials such as conjugated polymers, graphene, carbon nanotubes (CNTs) and metal nanoparticles (Minteer, et al., Materials Today 2012, 15 (4), 166-173).

However, the relatively low power output of current EFCs resulting from misaligned energy levels between the enzyme and the conducting material and their poor operational stability related to the overtime denaturation and activity drops of the enzyme have restricted any practical applications of these devices (Aghahosseini, et al., Nanochemistry Research 2016, 1 (2), 183-204; Kwon, et at, Nature Communications 2018, 9 (1), 4479). In addition, weak bio-electronic coupling and limited mass transport restrain the performance of the EFCs (Kwon, et al., Nature Communications 2018, 9 (1), 4479; Wen and Eychmüller, Small 2016, 12 (34), 4649-4661). Most EFCs contain electron mediators which complicates their fabrication and sacrifices the stability.

There remains a need for improved EFCs for providing power.

It is therefore an object of the present invention to provide improved enzymatic biofuel cells.

It is another object of the present invention to provide methods of making the enzymatic biofuel cells.

It is yet another object of the present invention to provide methods of using the enzymatic biofuel cells.

### SUMMARY OF THE INVENTION

Improved enzymatic biofuel cells (EFCs), methods of making and using thereof, are provided.

The EFC includes a bioanode, a biocathode, and an electrolyte solution. Typically, the EFC is electron mediator-free (i.e. does not include an electron mediator).

The bioanode includes the following: (a) a conductive substrate; (b) one or more n-type polymers; and (c) one or more enzymes. Optionally, the bioanode also includes a coating that encapsulate the one or more n-type polymers and the one or more enzymes on the conductive substrate. The one or more n-type polymers are typically semiconducting or conducting polymers.

The biocathode includes the following: (a) a conductive substrate; and (b) one or more p-type polymers. The one or more p-type polymers are typically semiconducting or conducting polymers.

The electrolyte solution contains one or more metabolites capable of reacting with the one or more enzymes in the bioanaode, and is in electrical communication with the bioanode and the biocathode. In one preferred embodiment the enzyme is glucose oxidase (GOx) and the metabolite is glucose. The bioanode is electrically connected to the biocathode. The biofuel cell generates power when the metabolites react with the enzymes to produce electrons (anodic reaction); the electrons are directed through an electrical circuit to the biocathode where an oxidant is reduced to water (cathodic reaction).

In some embodiments, the EFC includes a membrane that separates the bioanode and the biocathode. The membrane divides the EFC into two compartments such that the anodic reaction and cathodic reaction are separated.

The structure of the n-type polymer facilitates interactions of the enzyme (i.e. the active site of the enzyme) with the polymer, and allows efficient electrical communication between the enzyme and the polymer. This way, the electrons generated from the enzymatic reaction are transferred efficiently from the redox center to the polymer backbone. In some preferred embodiments, the n-type polymer is naphthalene-1,4,5,8-tetracarboxylic-diimide-bithiophene (NDI-T2) polymer with 90% glycol chain percentage (P90).

Also provided are methods of making EFC which includes a bioanode comprising (a) a conductive substrate; (b) one or more n-type polymers; and (c) one or more enzymes and a biocathode including the following: (a) a conductive substrate; and (b) one or more p-type polymers. The EFC is made by; (1) contacting one or more n-type polymer with a conductive substrate and at least one or more enzymes to form a bioanode; (2) contacting conductive substrate with one or more p-type polymers to form a biocathode; (3) electrically connect the bioanode and the biocathode; and (4) contacting the connected bioanode and biocathode with an electrolyte solution contain one or more metabolites/substrate reactive with the one or more enzymes. Optionally, the biocathode is further functionalized with one or more enzymes that reduce oxygen. In some preferred embodiments, the distance between the anode and cathode is between 0.1 to 3 cm.

The biofuel cells disclosed herein can be utilized as an implanted device or power sources for implantable devices, including, but not limited to, pacemakers, insulin pumps, sensors or an array of sensors. In another embodiment, the biofuel cell can be used in vitro for powering portable devices and wearable electronics, including, but not limited to, glucometer, sensor, or an array of sensors for continuous monitoring of metabolites, ions, pH, or temperature. In some embodiments, the biofuel cell can be used for powering electronics, such as LED. In yet another embodiment, the biofuel cell can be utilized as a self-powered sensor that spontaneously and continuously monitor chemicals such as multiple metabolites that react with the enzymes.

### BRIEF DESCRIPTION OF THE DRAWINGS

FIG. 1 is a graph showing cyclic voltammogram of a P-90 electrode in the absence and presence of GOx. The marked points in the P-90 curve denote the redox peaks. As glucose concentration increases, the spectrum changes further. The scan rate is 50 mV s⁻¹ and the solution is PBS. The arrow indicates the scan direction.

FIGS. 2A-2D are graphs showing cyclic voltammogram of P-90/GOx system recorded in PBS in air and inert atmosphere in the absence (FIG. 2A) and in the present of 250 μM glucose (FIG. 2B), 500 μM glucose (FIG. 2C), and 5 mM glucose (FIG. 2D), respectively.

FIG. 3 is a graph showing QCM-D measurements tracking the interactions between GOx and the two polymer films differing with their glycol content (P-90; ratio of glycol:alkyl side chains is 90:10, and P-0; ratio of glycol:alkyl side chains is 0:100) during two stages: (1) when the enzyme was injected into the PBS solution (+GOx) and (2) when the enzyme-exposed P-90 film was rinsed with PBS (Rinse).

FIG. 4 is a schematic showing possible events that take place upon oxidation of glucose by GOx at the P-90 interface.

FIG. 5 is a graph showing UV-VIS spectrum of a P-90 film in the absence and presence of a doping bias (0.5 V vs. Ag/AgCl) in PBS.

FIG. 6A is a graph showing the thin film UV-VIS spectrum of P-90 upon adsorption of the enzyme and the enzymatic reaction with 1 mM of glucose. FIG. 6B is a graph showing UV-VIS spectrum of a P-90/GOx film in PBS before and after the addition of glucose (1 mM). The film was subject to a constant doping potential at 0.5 V vs Ag/AgCl during the course of the experiments. FIG. 6C is a graph showing UV-VIS spectrum of a P-90/GOx film measured in PBS with and without the addition of 1 mM of H₂O₂.

FIG. 7A is a graph showing Raman spectrum of a P-90 film subject to increasing doping potentials, from 0 V to +1 V. After 1 V, the film was de-dedoped by applying 0 V vs Ag/AgCl (“0 V, reverse”). FIG. 7B is a graph showing Raman spectrum of a P-90 film at 0 V (de-doped) as well as when it is biased at increasing doping potentials (0.7 V-1 V). FIG. 7C is a magnified view of FIG. 7B from 1250 cm⁻¹ to 1500 cm⁻¹.

FIG. 8A is a graph showing Raman spectrum of a P-90 film compared to that of P-90/GOx film. FIG. 8B is a magnified view of FIG. 8A showing region between 1350 cm⁻¹ and 1750 cm′.

FIG. 9A is a graph showing Raman spectra of a pristine P-90 film (0 V, reduced P-90 film (0.7 V) and P-90/GOx film in the presence of 1 mM of glucose (0.7 V). FIG. 9B is a magnified view of FIG. 9A from about 1250 cm⁻¹ to about 1500 cm⁻¹.

FIG. 10 is a graph showing schematic of the EFC comprising P-90/GOx at the anode and p(EDOT-co-EDOTOH) at the cathode.

FIG. 11 is a graph showing reactions occurring during the operation of the EFC.

FIG. 12A is a schematic showing the experimental set-up for the electrodeposition of p(EDOT-co-EDOTOH). FIG. 12B is a graph showing chronoamperometry under 1 V vs. Ag/AgCl at 25° C. during electropolymerization of the EFC cathode.

FIG. 13A is a graph showing chronoamperometry of the p(EDOT-co-EDOTOH) cathode under various atmospheres. The gases were introduced by bubbling the solution for 15 minutes. Afterwards, the gas tubing was held above the solution to shield it from the outside atmosphere. The measurement was performed at 0 V vs. V_{OC}. FIG. 13B is a graph showing cyclic voltammogram of p(EDOT-co-EDOTOH) cathode recorded in air and in O₂ saturated buffer by using a rotating disk electrode. Scan rate is 10 mVs⁻¹ and rotation rate is 10 Hz (600 rpm). Arrow indicates the scan direction. FIG. 13C is a graph showing reciprocal of current density of p(EDOT-co-EDOTOH) as a function of the rotation speed.

FIG. 14A is a graph showing extended cyclic voltammogram of the EFC comprising a P-90/GOx anode and a p(EDOT-co-EDOTOH) cathode, fueled by 1 mM of glucose in PBS. Scan rate is 5 mV·s⁻¹ and arrow indicates the scan direction. The dotted lines depict the onset potential for the reduction and oxidation reactions, respectively. The difference between the onset potentials gives the theoretical open circuit voltage of the cell (Luz, et al., ChemElectroChem 2014, 1 (11), 1751-1777). FIG. 14B is a magnified view of the potential window from 0 V to 0.6 V.

FIGS. 15A-15B are graphs showing open circuit voltage (OCV) of P-90/GOx based EFCs with (FIG. 15A) or without (FIG. 15B) comprising different cathodes, Pt or p(EDOT-co-EDOTOH). Measurements are performed in PBS containing 1 mM of glucose.

FIG. 16 is a graph showing stability of EFC cathode p(EDOT-co-EDOTOH) measured in PBS over 100 cycles by cyclic voltammetry. Scan rate is 100 mV·s⁻¹. Arrows indicate the scan direction. Cᵣₑₜ represents capacitance retention.

FIGS. 17A-17D are graphs showing electrochemical characterization of the bioanode performed in PBS containing 10 mM of glucose. FIGS. 17B-17C show the current density as a function of the scan rate for the two distinct redox couples marked in the CV curve shown in FIG. 17A. The linear relationship between current density and scan rate corroborates a surface-controlled process. FIG. 17D shows Laviron plots for the oxidation and reduction reactions. The peak potentials are plotted against the logarithm of the scan rate. At high scan rates, the linear relationship between ΔE_{PP} and ln(ν) yields the electron transfer coefficient (α).

FIG. 18 is a graph showing stability of EFC bioanode P-90/GOx measured in PBS over 100 cycles by cyclic voltammetry. Scan rate is 100 mV·s⁻¹. Arrows indicate the scan direction. J_{ox.ret} represents oxidation current retention, and J_{Red.ret} represents reduction current retention.

FIG. 19A is a graph showing open circuit voltage (OCV) of the EFC containing P-90 alone and when it is functionalized with GOx as well as when the cell is fueled by 1 mM of glucose in PBS. FIG. 19B is a graph showing power output of the EFC before anode functionalization with GOx and after anode functionalization with GOx. FIG. 19C is a graph showing power output of the EFC when the anode is functionalized with GOx and in the presence of selected concentrations of glucose as a function of current density. The data were obtained by varying the circuit load. The measurements were performed in PBS (pH 7.2). The EFC includes a Nafion membrane.

FIGS. 20A-20C are graphs showing the electrochemical characterization for the membrane-less, all polymer biofuel cell. FIG. 20A is a graph showing dependence of cell voltage and power on current density of the EFC fed with 10 mM of glucose solution. The measurements were acquired with an external resistor (1 kΩ-10 MΩ). FIG. 20B is a graph showing OCV and MPD dependence on glucose concentration. FIG. 20C is a graph showing comparison of the MPD of EFCs prepared with and without a Nafion membrane as a function of glucose concentration. The error bars show the standard deviation of the mean value of OCV and MPD values for three independent measurements.

FIG. 21A is a graph showing Nyquist plots of the EFC with and without Nafion membrane. FIG. 21B shows the equivalent circuit of the EFC. The circuit contains three parts. The first part is the ohmic component which accounts for the electrolyte resistance between the anode and cathode. The other two parts model the anode (P-90/GOx) and the cathode (p(EDOT-co-EDOTOH)) compartments of the cell. Both anode and cathode polymer films include a capacitor that represents the double layer charging at the surface of the polymer and a resistor that depicts the interfacial resistance between electrode and electrolyte (Choi, et al., Sensors and Actuators A: Physical 2012, 177, 10-15; Ye, et al., International Journal of Hydrogen Energy 2013, 38 (35), 15710-15715). The overall resistance of the cell is the sum of these three parts (R_{membrane-free}=600 vs. R_{Nafion 117}=787Ω).

FIG. 22 is a graph showing the performance of a membrane-free EFC comprising an electrochemically doped P-90. The P-90 film is functionalized with GOx and placed in PBS as the working electrode of a three-electrode system where a Pt coil is the counter electrode and Ag|AgCl electrode is the reference electrode. The P-90 electrode is also connected to the p(EDOT-co-EDOTOH) cathode. The power output of this EFC is markedly higher than the pristine EFC in the presence of 1 mM glucose, determined using an external resistor (1 kΩ-10 MΩ).

FIGS. 23A-23C are graphs showing linear sweep voltammogram of the cell (FIG. 23A) with and (FIG. 23C) without a Nafion membrane for the enzymatic reaction conditions (1 mM of glucose) along with the 1^{st} derivative of the voltammogram. The current was collected at a scan rate of 1 mV·s⁻¹ to ensure steady-state. The inflection point defines the voltage at which the rate of increase of the redox reactions in the cell reaches a maximum. The current value at the inflection point is used to calculate the power output of the cell for the stability studies. FIG. 23B is a graph showing the absorbance of H₂O₂ in the cell as a function of H₂O₂ concentration to determine the amount of H₂O₂ in the cell at 0.7 and 1 V using a colorimetric assay.

FIGS. 24A-24C are graphs showing relative voltage (OCV/OCV₀) measured at open circuit conditions and relative power density (PD/DP₀) of the EFC (with a Nafion membrane) fueled by 1 mM of glucose in PBS. The PD values are determined at an anodic bias (e.g. 1 V, see FIG. 23A-23C). FIG. 24A is a schematic showing the EFC where a membrane separates the anode and cathode. FIG. 24B is a graph showing OCV/OCV₀ and PD/DP₀ when the enzyme is adsorbed on the anode at day 0 and kept as is until day 30. FIG. 24C is a graph showing OCV/OCV₀ and PD/DP₀ when the enzyme is replenished before each measurement and measurements continued for 50 days. The anode and cathode are separated with a Nafion membrane (123 μm) as depicted in the top schematic. FIG. 24D is a schematic showing the EFC where a coating is on the surface of the bioanode to encapsulate the polymer and enzymes. FIG. 24E is a graph showing OCV/OCV₀ and PD/DP₀ when the enzyme is adsorbed on the anode at day 0 and kept as is until day 30. A Nafion film was cast on top of GOx functionalized anode as depicted in the top schematic and used as the encapsulation layer. The error bars show the standard deviation of the mean value of OCV and PD values for four independent measurements.

FIGS. 25A-25B are graphs showing Relative voltage (OCV/OCV₀) and relative power (PD/DP₀) in membrane-free EFC measured at open circuit conditions and fueled by 1 mM of glucose in PBS, where the enzyme is adsorbed on the anode at day 0 and kept as is until day 30 (FIG. 25A) or the enzyme is replenished before each measurement (FIG. 25B).

FIGS. 26A-26B are graphs showing performance of the EFC (with Nafion membrane separator) operated with a broad range of glucose concentrations in PBS (FIG. 26A) and saliva (FIG. 26B).

FIG. 27A is a schematic showing the circuit connection of the polarized capacitor. FIG. 27B is a graph showing the output voltage of the EFC charging a capacitor (100 μF) as a function of time. With prolonged charging, an array of three EFCs in series was introduced.

FIG. 28A is a schematic showing the OECT biosensor the connection of the EFC cell with the OECT biosensor. FIG. 28B is a graph showing real-time response of a PEDOT:PSS OECT gated with a membrane-free EFC that is fueled by a range of glucose concentrations. The gate electrode was disconnected from the electrolyte while changing the glucose concentration in the EFC cell.

FIG. 29A is a schematic showing the electrical circuit of an accumulation mode OECT powered by an EFC, where the V_{G} and V_{D} are supplied by the biofuel cells with an output voltage of −0.15 V and −0.07 V, respectively. FIG. 29B is a graph showing the OECT switched ON and OFF by reversing the polarity of the gate voltage (by swapping the anode/cathode of the biofuel cell connected to the gate electrode). The EFCs are fed with a constant concentration of aqueous glucose as fuel.

FIG. 30 is a schematic showing an exemplary microfluidic configuration that incorporates the biofuel cell.

### DETAILED DESCRIPTION OF THE INVENTION

### I. Definitions

As used herein, a “biofuel cell” or “enzyme-based biofuel cell” includes an anode and a cathode, which are electrically connected but separated to avoid an electrical short. Preferably, the anode and cathode are kept at a distance. In one embodiment, the distance between the electrodes is kept at 1 cm. A biofuel cell utilizes a metabolite and an enzyme which catalyzes a reaction of the metabolite. In one embodiment, a “biofuel cell” utilizes a metabolite and an enzyme to catalyze the oxidation of the metabolite. The terms “biofuel cell” and “enzyme-based biofuel cell” are used interchangeably throughout the instant disclosure. In one embodiment, the biofuel cell disclosed herein may be used in applications that require an electrical supply, such as, but not limited to electronic devices and equipment, toys, internal medical devices, and electrically powered vehicles. In another embodiment, the biofuel cell disclosed herein may be implanted into a living organism of a subject, wherein the metabolite is derived from the organism and the biofuel cell powers itself or a device implanted in the living organism.

As used herein, the term “anode” or “bioanode” refers to an anode containing an enzyme that catalyzes the oxidation of a metabolite. A bioanode provides a source of electrons for an electrical circuit or electrical potential. In one embodiment, a preferred bioanode also contains a conductive substrate and polymeric materials. The terms “anode” and “bioanode” are used interchangeably throughout the instant disclosure.

As used herein, the term “cathode” or “biocathode” refers to a cathode at which an oxidant is reduced in the presence of electrons. In a preferred embodiment, the oxidant is oxygen. In one embodiment, the biocathode contains an enzyme, such as laccase, which catalyzes the reduction of oxygen. In another embodiment, a preferred biocathode also contains a conductive substrate and polymeric materials. In the most preferred embodiment, the biocathode contains a conductive substrate and polymeric materials but not enzymes. The terms “cathode” and “biocathode” are used interchangeably throughout the instant disclosure.

As used herein, the term “conductive substrate” refers to a substance capable of conducting an electric current and used to support the polymeric material of a biofuel cell electrode. The conductive substrate can be organic or inorganic in nature as long as it is able to conduct electrons through the material. The conductive substrate can be a polymeric conductor, a metallic conductor, a semiconductor, a carbon-based material, a metal oxide, or a modified conductor.

As used herein, the term “polymer” or “polymeric material” refers to a polymer capable of accepting or donating electrons from or to a compound, resulting in the oxidation or reduction, respectively, of the compound and the generation of free electrons for transferring into an electric circuit. The term “n-type polymer” or “n-type semiconducting polymer” refers to a polymer capable of accepting electrons and stabilizing electrons on its backbone. A preferred “n-type semiconducting polymer”, P90, is based on an NDI-T2 copolymer, which has a backbone comprising a highly electron-deficient naphthalene-1,4,5,8-tetracarboxylic diimide (NDI) repeat unit and an electron-rich unsubstituted bithiophene repeat unit (T2). (Giovannitti, et al., Chemistry of Materials, 30:2945-2953 (2018); Pappa, et al., Science Advances, 4:eaat0911 (2018)). The terms “n-type polymer” and “n-type semiconducting polymer” are used interchangeably throughout the instant disclosure. The term “p-type conducting polymer” refers to a polymer capable of donating electrons. A preferred “p-type conducting polymer”, PEDOT:PEDOT-OH, is a mixture of poly(3,4-ethylenedioxythiphene) (PEDOT) and poly(hydrooxymethyl 3,4-ethylenedioxythiphene) (PEDOT-OH). The terms “polymer” and “polymeric material” are used interchangeably throughout the instant disclosure.

As used herein, the term “enzyme” refers to a protein that functions as a catalyst in a chemical reaction. Enzymes include, but are not limited to glucose oxidase, glucose dehydrogenase, alcohol dehydrogenase, aldehyde dehydrogenase, formate dehydrogenase, formaldehyde dehydrogenase, lactic dehydrogenase, lactose dehydrogenase, lactate oxidase, cholesterol oxidase, tyrosinase, and pyruvate dehydrogenase. Preferred enzymes include oxidase which catalyzes the oxidation of a metabolite, such as glucose oxidase (GOx). Enzymes useful for biocathodes include oxygen reductase, such as laccase and bilirubin oxidase.

As used herein, the term “metabolite” means any compound that has stored energy. Preferred metabolites are carbon-based compound that has stored energy. Metabolites include but are not limited to nucleic acids, carbohydrates, alcohols, fatty acids and other hydrocarbons, ketones, aldehydes, amino acids, and proteins. The “metabolite” may be a biological compound within an organism. Preferred metabolites are carbohydrates, which include glucose, glucose-2, D-glucose, L-glucose, and glucose-6-phosphate.

As used herein, the term “electrolyte solution” refers to a solution that contains ions, atoms, or molecules that have lost or gained electrons, and is electrically conductive. The electrolyte solution is in electrical communication with the bioanode and the biocathode. The electrolyte solutions contains one or more metabolites.

As used herein, the term “ambient oxygen concentration” refers to the concentration of molecular oxygen dissolved in a liquid such as electrolyte solution or biological fluids under ambient conditions (atmospheric pressure, T=293 K) (Zebda, et al., Scientific Reports, 3:1516 (2013)).

As used herein, the term “physiological relevant concentration” refers to the concentration of the external or internal milieu that may occur in nature for that organism or cell system (Bruen, et al., Sensors, 17:1866 (2017)).

As used herein, the term “stability” refers to the biofuel cell's capability to preserve at least 20% of its original maximum power density (MPD). Improved stability is used to refer to the biofuel cell's capability to preserve at least 30% of its original MPD for at least 30 days.

As used herein, the term “open circuit potential (OCP)” refers to the difference between the thermodynamic potential of the oxidation of one compound at the anode and the reduction of another compound at the cathode. In a preferred embodiment, OCP refers to the difference between the thermodynamic potentials of the oxidation of the metabolite at the anode and the reduction of oxygen at the cathode.

As used herein, the term “sensor” refers to a device that detects or measures an event or a change of a physical property of an analyte, and records, indicates, or responds to the event or change. In one embodiment, the sensor can measure or sense metabolites, ions, pH, or temperature. In some embodiments, the sensor can measure or sense one or more analytes.

As used herein, the term “polar group” refers to a group in which the bond dipoles present do not cancel each other out and thus results in a molecular dipole.

As used herein, the term “alkylene glycol” or “glycol” refers to ethylene oxide, propylene oxide, or copolymers of ethylene oxide or propylene oxide. The terms “alkylene glycol” or “glycol” are used interchangeably throughout the instant disclosure.

### II. Enzyme-Based Biofuel Cell

The Examples below demonstrate for the first time the use of an n-type semiconducting polymer as an anode material in an electron mediator-free and optionally, in some preferred embodiments, a membraneless glucose-oxygen biofuel cell configuration as an electrochemical energy powering source.

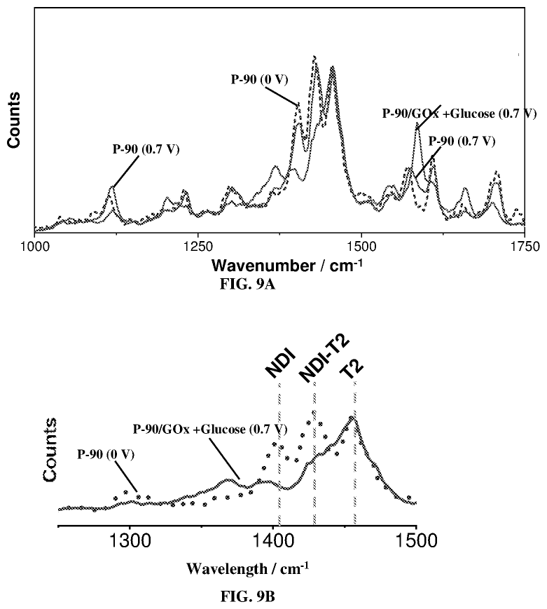

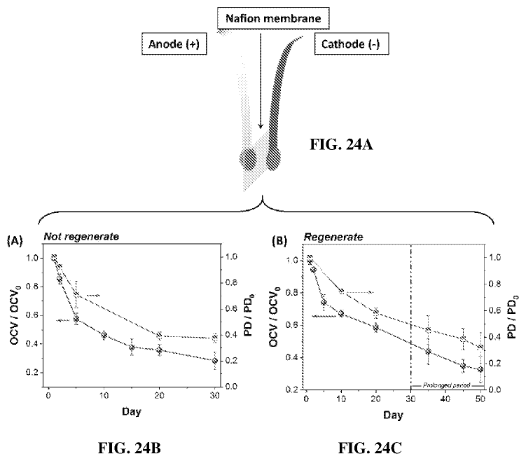

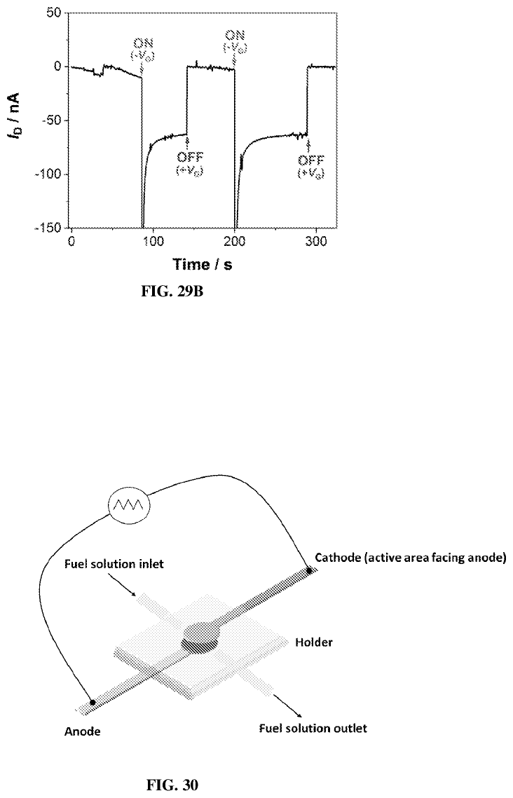

Enzyme-based biofuel cells enable energy generation via chemical transformation of metabolites. In some embodiments, the metabolites present in organic tissues, such as glucose and oxygen, serve as ideal fuel sources as they are readily available in all biological fluids and are easily replenished (Slaughter, et al., Journal of Biochips & Tissue Chips, 5(1):1 (2015)). Enzyme-based biofuel cells include a bioanode, a biocathode, and electrolyte solution containing one or more metabolites (FIG. 10). The bioanode and biocathode are connected electronically and placed apart to avoid shorts. The bioanode and the biocathode can be kept at a distance between 0.1 to 3 cm. In one embodiment, the distance between the bioanode and the biocathode (i.e., the inter-electrode gap) is kept at 0.5 cm. The inter-electrode gap can be varied to mitigate ohmic resistance losses. Optionally, the EFC includes a membrane that separates the bioanode and the biocathode (see, for example, FIG. 24A). The membrane divides the EFC into two compartments such that the anodic reaction and cathodic reaction are carried out separately. The electrolyte solution is in electrical communication with the bioanode and the biocathode. In some embodiments, the biofuel cell can be incorporated into a microfluidics configuration (FIG. 30).

Typically, the metabolites react with the enzyme to produce the oxidized form of the metabolite and produces electrons, resulting in doping of the n-type polymers via electron transfer at the bioanode. The electrons are transferred from the external circuit to the cathode, reducing an oxidant to water. The reduction of enzyme is reversible so enzymes are not consumed.

Optionally, the redox reactions can be irreversible if an electron mediator is added to provide additional reactant. A conductive substrate and an enzyme can be used wherein an electron mediator in contact with the bioanode is able to transfer electrons between its oxidized and reduced forms at the bioanode.

The disclosed EFCs can generate a maximum power density (MPD) of at least 0.1 μW·cm⁻², at least 0.2 μW·cm⁻², at least 0.3 μW·cm⁻², at least 0.4 μW·cm⁻², at least 0.5 μW·cm⁻², at least 0.6 μW·cm⁻², at least 0.7 μW·cm⁻², at least 0.8 μW·cm⁻², at least 0.9 μW·cm⁻², at least 1 μW·cm⁻², at least 1.1 μW·cm⁻², at least 1.2 μW·cm⁻², at least 1.3 μW·cm⁻², at least 1.4 μW·cm⁻², at least 1.5 μW·cm⁻², at least 1.6 μW·cm⁻², at least 1.7 μW·cm⁻², at least 1.8 μW·cm⁻², at least 1.9 μW·cm⁻², at least 2 μW·cm⁻², at least 2.1 μW·cm⁻², at least 2.2 μW·cm⁻², at least 2.3 μW·cm⁻², at least 2.4 μW·cm⁻², or at least 2.5 μW·cm⁻². In some embodiments, the disclosed EFC does not include a membrane, and can generate a MPD of at least 0.4 μW·cm⁻². In some embodiments, the disclosed EFC includes a membrane, and can generate a MPD of up to 2.7 μW·cm⁻².

The disclosed EFCs can generate an open circuit potential of at least 0.1 V, at least 0.15 V, at least 0.18 V, at least 0.19 V, at least 0.2 V, at least 0.25 V, at least 0.26 V, at least 0.27 V, at least 0.28 V, at least 0.29 V, at least 0.3 V. In some embodiments, the disclosed EFC does not include a membrane, and can generate an OCP of at least 0.15 V or at least 0.18 V. In some embodiments, the disclosed EFC includes a membrane, and can generate an OCP of up to 0.31 V.

In some embodiments, the disclosed EFC is membrane-free and can generate a MPD of at least 0.2 μW·cm⁻², at least 0.3 μW·cm⁻², or at least 0.4 μW·cm⁻², and an OCP of at least 0.15 V or at least 0.18 V. In some embodiments, the disclosed EFC includes a membrane and can generate a MPD of at least 0.15 V or at least 2 μW·cm⁻², at least 2.5 μW·cm⁻², or up to 2.7 μW·cm⁻², and an OCP of at least 0.25 V, at least 0.3 V, or up to 0.31 V.

This simple and functionalization-free biofuel cell displays superior stability. For example, the disclosed EFC can preserve at least 15%, at least 20%, at least 30%, at least 35%, at least 40%, or at least 45% of its original maximum power density (MPD) after at least 30 days, at least 35 days, at least 40 days, at least 45 days, or at least 50 days. For example, the disclosed EFC can preserve at least 45% of its original MPD after 30 days and/or preserve at least 15% of its original MPD after 50 days. In some embodiments, the disclosed EFC can preserve at least 20% or 30% of its original MPD after 30 days. Optionally, the disclosed EFC can preserve at least 20%, at least 30%, at least 35%, at least 40%, at least 45%, at least 50%, at least 55%, at least 60%, at least 65%, or at least 70% of its original open circuit potential (OCP) after at least 30 days, at least 35 days, at least 40 days, at least 45 days, or at least 50 days. For example, the disclosed EFC can preserve at least 60% of its original OCP after 30 days and/or at least 30% of its original OCP after 50 days. In some embodiments, the disclosed EFC can preserve at least 70% of its original OCP after 30 days.

Optionally, the enzymatic biofuel cell, in its simplest design: (i) avoids the tedious processes of enzyme immobilization, (ii) does not need an electron mediator, (iii) is simple and scalable, (iv) can be utilized as a self-powered multi-analyte sensors, (v) the enzymatic biofuel cell is stable for up to 50 days, (vi) it does not need oxygen reductase at the cathode, (vii) it performs at ambient oxygen concentration and physiological pH, and (viii) the electrodes are made of conducting polymers coated on a thin layer of metal, which reduces the cost related to conventional electrodes made of thick and expensive catalytically active metals. In some embodiments, the physiologically relevant concentration of glucose is between 0.1 μM and 10 mM, inclusive. In some embodiments, the physiological relevant concentration of glucose is between 0.1 μM and 1 mM, inclusive. The physiological concentration of glucose in different body fluids is disclosed in Bruen, et al., Sensors, 17:1866 (2017).

Additionally, the disclosed biofuel cells advantageously operate with ambient oxygen. In some embodiments, the ambient oxygen concentration for air saturated liquid is between 1 μM to 300 μM. In a preferred embodiment, the ambient oxygen concentration for air saturated liquid is 267 μM (Tao, et al., Biophysical Journal, 96(7):2977-2988 (2009)). By contrast, previously disclosed biofuel cells require saturation of the electrolyte solution with oxygen or the use of an enzyme such as an oxygen reductase to catalyze an oxygen reduction reaction (Christwardana, et al., Scientific Report, 6:30128 (2016).

A. Bioanode

Generally, the bioanode contains elements that effect the oxidation of metabolites wherein electrons are released and directed to an external electrical circuit. The external electrical circuit is in contact with a cathode where an oxidant is reduced. This flow of electrons from the enzymatic oxidation reaction to the components of the bioanode through an electrical circuit to a cathode provides a source of energy.

In a biofuel cell, the reaction that occurs at the anode is the oxidation of a compound such as a metabolite with a concurrent release of electrons; the electrons are directed through an electrical connection to the cathode where an oxidant is reduced to water. The biofuel cell disclosed herein provides an energy source for an electrical device external to the biofuel cell. To facilitate the oxidation of the compounds such as metabolites, the bioanode contains a conductive substrate, one or more n-type polymers, and one or more enzymes. In the most preferred embodiment, the n-type polymer is P90.

In some embodiment, the bioanode contains a conductive substrate, one or more polymeric materials, and one or more enzymes. Optionally, the bioanode contains only one enzyme (i.e., one type of enzyme), such as glucose oxidase. Optionally, the bioanode further contains a coating that encapsulate the one or more polymeric materials and/or one or more enzymes on the conductive substrate. The coating may increase enzyme stability when the EFC is intended for long term use, such as at least 30 days. When a coating is included in the bioanode, the EFC is preferably a membrane-free device, i.e. does not include a membrane that separates the cathode and anode. In some embodiments, the bioanode optionally further contains an electron mediator. Optionally, the bioanode does not include an electron mediator. An electron mediator can be absent from the bioanode when the bioanode contacts an n-type semiconducting polymer that is capable of interacting with the enzymes and promoting electron transfer from the oxidation reactions to the bioanodes. Optionally, the bioanode does not contain a carbon-based material. For example, the bioanode does not contain carbon nanotubes. Optionally, the bioanode contains a carbon-based material only as the conductive substrate.

The above-identified components of the bioanode are adjacent to one another; meaning they are physically or chemically connected by appropriate means. In one embodiment, the components are physically and chemically connected by placement into a solution with an electrical connection between them. In a preferred embodiment, the component are physically connected by coating such as by spin-coating, drop-casting, or electropolymerization, or otherwise deposing the individual components on the conductive substrate. The components can be deposited separately, e.g. in layers, or they can be integrated into one deposition layer.

1. Conductive Substrate

The conductive substrate is a substance capable of conducting an electric current. It is used to support the polymeric material and enzymes of a biofuel cell electrode. The conductive substrate can be organic or inorganic in nature, as long as it is able to conduct electrons through the material. The conductive substrate can be a polymeric conductor, a metallic conductor, a semiconductor, a carbon-based material, a metal oxide, or a modified conductor.

In some embodiments, the conductive substrate is made of a metallic conductor. Suitable metallic conductors include but are not limited to gold, chromium, platinum, iron, nickel, copper, silver, stainless steel, mercury, tungsten and other metals suitable for electrode construction. The metallic conductor can be a metal alloy which is made of a combination of metals disclosed above. In addition, conductive substrates which are metallic conductors can be constructed of nanomaterials made of gold, cobalt, diamond, and other suitable metals.

In other embodiments, the conductive substrate is made from carbon-based materials. Exemplary carbon-based materials are conducting polymers (in the form of films or fibers) carbon cloth, carbon paper, carbon screen printed electrodes, carbon paper, carbon black, carbon powder, carbon fiber, singe-walled carbon nanotubes, double-walled carbon nanotubes, multi-walled carbon nanotubes, carbon nanotube arrays, diamond-coated conductors, glassy carbon and mesoporous carbon. In addition, other exemplary carbon-based materials are graphene, graphite, uncompressed graphite worms, delaminated purified flake graphite, high performance graphite and carbon powders, highly ordered pyrolytic graphite, pyrolytic graphite, and polycrystalline graphite.

The conductive substrate can be a semiconductor. Suitable semiconductors are prepared from silicon and germanium, which can be doped (i.e., the intentional introduction of impurities into an intrinsic semiconductor for the purpose of modulating its electrical and structural properties) with other elements. The semiconductors can be doped with phosphorus, boron, gallium, arsenic, indium or antimony, or a combination thereof.

Other conductive substrate can be metal oxides, metal sulfides, main group compounds, and modified materials. Exemplary conductive substrates of this type are nanoporous titanium oxide, tin oxide coated glass, cerium oxide particles, molybdenum sulfide, boron nitride nanotubes, aerogels modified with a conductive material such as gold, solgels modified with conductive material such as carbon, ruthenium carbon aerogels, and mesoporous silicas modified with a conductive material such as gold.

In another preferred embodiment, the conductive substrate contains one or more conducting materials. In embodiments where the conductive substrate containing two or more conducting materials, the first conducting material can be a conducting polymer and a second conducting material can be a material disclosed above. The conducting polymers include but are not limited to poly(fluorine)s, polyphenylenes, polypyrenes, polyazulenes, polynaphthalenes, poly(pyrrole)s, polycarbozoles, polyindoles, polyzaepines, polyanilines, poly(thiophene)s, poly(3,4-ethylenedioxythiophene), poly(p-phenylene sulfide), poly(acetylene)s, poly(p-phenylene vinylene), and polyimides. The second conducting material can be sputter coated on top of the conducting polymer, and the aggregate of the two makes up the conductive substrate. A preferred conductive substrate is a Kapton (polyimide) film sputter coated with Cr/Au.

2. Polymeric Materials

The polymeric material of the bioanode is any polymer that is capable of accepting (n-type) electrons from a compound, resulting in the oxidation of the compound and the generation of free electrons for transferring into an electric circuit. The n-type polymer is a polymer capable of accepting electrons and stabilizing electrons on its backbone. Exemplary n-type polymers include N2300, P(NDI-T2), poly(diketopyrrolopyrrole) (DPP), polybenzimidazobenzophenanthroline (BBL), poly(benzimidazobenzophenanthroline), poly(2,5-di(3,7-dimethyloctyloxy)cyanoterephthalylidene), poly(2,5-di(hexyloxy)cyanoterephthalylidene), poly(5-(3,7-dimethyloctyloxy)-2-methoxy-cyanoterephthalylidene), poly(2,5-di(octyloxy)cyanoterephthalylidene), and poly(5-(2-ethylhexyloxy)-2-methoxy-cyanoterephthalylidene). Optionally, the.

In some embodiments, the n-type polymer is a structure-modified polymer to introduce one or more additional polar groups. For example, DPP polymers can be modified to incorporate lysine side chains. Exemplary polar groups include molecules with a single hydrogen, such as OH, molecules with at least one OH at one end, such as alcohol and alkylene glycol, and molecules with an N at one end, such as ammonia. In a preferred embodiment, the polar group is an alkylene glycol.

In the most preferred embodiment, the n-type polymer is P90, which is based on an NDI-T2 copolymer having a backbone comprising a highly electron-deficient naphthalene-1,4,5,8-tetracarboxylic diimide (NDI) repeat unit and an electron-rich unsubstituted bithiophene repeat unit (T2). (Giovannitti, et al., Chemical Materials, 30:2945-2953 (2018); Pappa, et al., Science Advances, 4:eaat0911 (2018)). An exemplary structure of P90 is shown below. The side chains on the diimide unit are a 90:10 randomly distributed ratio of polar glycol and nonpolar branched alkyl groups. Generally, the ratio of polar groups to nonpolar groups can be optimized to ensure solubility of the n-type polymer in polar solvents (Giovannitti, et al., Chemical Materials, 30:2945-2953 (2018)).

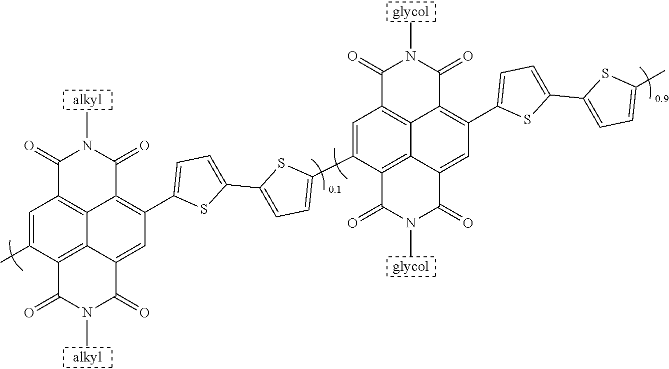

The polar groups such as the glycol side chains of P90 are envisaged to serve the dual role of (i) providing polar groups for the enzyme to interact with and (ii) enhancing the polymer's water uptake capacity to promote electrochemical activity in aqueous media (Al-Ani, et al., Polymers, 9:343 (2017); Yang, et al., Journal of Polymer Science B Polymer Physics, 43:1455-1464 (2005); Giovannitti, et al., Proceedings of the National Academy of Sciences U.S.A., 113:12017-12022 (2016)). The polar groups in the polymer structure play a key role in the promotion of the entrapment of the enzyme and enzyme stabilization on the polymer surface.

3. Enzyme

An enzyme is necessary in the biofuel cell, to catalyze the oxidation of a compound. Accordingly, useful enzymes at the bioanode are enzymes involved in oxidation. Generally, naturally-occurring enzymes, man-made enzymes, artificial enzyme, and modified naturally-occurring enzymes can be used. In addition, engineered enzymes that have been engineered by natural or directed evolution can be used. An organic or inorganic molecule that mimics an enzyme's properties can be used in an embodiment of the present disclosure. Exemplary enzymes for use in a bioanode include glucose oxidase, glucose dehydrogenase, alcohol dehydrogenase, aldehyde dehydrogenase, formate dehydrogenase, formaldehyde dehydrogenase, lactic dehydrogenase, lactose dehydrogenase, lactate oxidase, cholesterol oxidase, tyrosinase, and pyruvate dehydrogenase. In a preferred embodiment, the enzyme is glucose oxidase.

Strategies for biofunctionalization of electrode surface with enzymes include: physical adsorption (i.e. spin-coating and drop-casting), covalent immobilization, cross-linking, affinity linking, and entrapment (Saboe, et at, Energy & Environmental Science, 10:14-42 (2017); Banica, Chemical Sensors and Biosensors: Fundamentals and Applications, John Wiley & Sons Ltd, United Kingdom (2012); Yates, et al., Chemistry: A European Journal, 24(47):12164-12182 (2018); Mateo, et al., Enzyme and Microbial Technology, 40(6):1451-1463 (2007); Sheldon, Advanced Synthesis & Catalysis, 349(8-9):1289-1307; Rocchitta, et al., Sensors, 16(6):780 (2016)). In a preferred embodiment, the enzyme is drop-casted on top of a conductive substrate coated with n-type polymers. Methods of drop casting enzyme solutions onto a surface are known in the art (Pappa, et al., Science Advances, 4(6):eaat0911 (2018)). The enzyme is efficiently anchored to the surface of the polymer because of the structure of the polymer. The polar groups of the polymer facilitate interactions of the enzyme with the polymer and bring the enzyme in close proximity to the surface of the conductive substrate (Inal, et al., ACS Applied Bio Materials, 1(5):1348-1354 (2018)). The polymer provides a suitable environment for enzyme to transfer electrons and produces a seamless polymer/enzyme interface. The efficient immobilization of the enzyme allows good electrical contact with the polymer, so the electrons generated from the enzymatic reaction can be transferred efficiently from the redox center to the polymer backbone. For example, the active sites of the enzyme and the copolymer have an interface which leads to an efficient electronic communication. At this bio-electronic interface, analyte oxidation increases the conductivity of the polymer by donating new electrons to the backbone, and this process proceeds without the aid of an external electron mediator. Efficient electrical communication between the polymer and the enzyme enables electron mediator-free electron transfer.

In a particularly preferred embodiment, biofunctionalization of the electrode surface does not require complex chemistry, i.e. covalent immobilization, cross-linking, or affinity linking.

4. Coating

Optionally, a coating is included in the bioanode (see, for example, FIG. 24D). The coating typically encapsulate the one or more polymeric materials and the one or more enzymes, and stabilize them on the surface of the conductive substrate. Any strategies for biofunctionalization of electrode surface with enzymes may be used to add the coating. For example, the coating is drop casted on the polymer and enzyme functionalized conductive substrate.

The coating is typically made of a cationic exchange material, such as polystyrene sulfonate and perfluorinated sulfonated ionomers. Exemplary materials for the coating include, but are not limited to, Nafion®, AQUIVION® (Solvay Sa Corporation, Brussels Belgium), or a combination thereof. For example, the coating is a Nafion® film.

5. Electron Mediator

Optionally, an electron mediator can be used in the biofuel cell. Optionally, the biofuel cell does not include an electron mediator. The electron mediator is a compound that can accept or donate electrons. The electron mediator can diffuse into the bioanode. Exemplary electron mediators are pyrroloquinoline quinone (PQQ), phenazine methosulfate, dichlorophenol indophenol, short chain ubiquinones, potassium ferricyan, or equivalents of each.

B. Biocathode

The biocathode carries out reduction reaction by utilizing electrons transferred though an electric circuit from the bioanode. In one embodiment, the biocathode contains a conductive substrate and one or more p-type polymers. In another embodiment, the biocathode optionally further contains an oxygen reductase. Optionally, the biocathode does not contain an enzyme, such as an oxygen reductase. Generally, the conductive substrates described above can be used. The advantages of the disclosed biofuel cell is its simplicity (with respect to fabrication) and at least the fact that despite not having an enzyme at the cathode, the disclosed device is still able to produce enough power for real applications. Therefore, in one preferred embodiment, the biocathode does not include an enzyme, for example, an oxygen reductase.

The p-type polymer of the biocathode is any polymer that is capable of donating electrons to a compound, resulting in the reduction of an oxidant. In a preferred embodiment, the p-type polymer is a robust oxygen reducer, and it reduces oxygen to water. Exemplary p-type polymers include, but are not limited to, poly(3,4-ethylenedioxythiphene) (PEDOT), poly(hydrooxymethyl 3,4-ethylenedioxythiphene) (PEDOT-OH), polystyrenesulfonate (PSS), F8BT, F8T2, J51, MDMO-PPV, MEH-PPV, PBDB-T, PBDTBO-TPD, PBDT(EH)-TPD, PBDTTT-C-T, PBDTTT-CF, PBTTPD, PBTTT-C14, PCDTBT, PCPDTBT, PDTSTPD, PffBT4T-20D, PffBT4T-C9C13, PFO-DBT, Poly([2,6′-4,8-di(5-ethylhexylthienyl)benzo[1,2-b;3,3-b]dithiophene] {3-fluoro-2[(2-ethylhexyl)carbonyl]thieno[3,4-b]thiophenediyl}, Poly(3-dodecylthiophene-2,5-diyl), Poly(3-hexylthiophene-2,5-diyl), Poly(3-octylthiophene-2,5-diyl), PSiF-DBT, poly(triaryl amine) (PTAA), PTB7, TQ1, and a combination thereof. In a preferred embodiment, the biocathode contains a mixture of two or more p-type polymers described above. In the most preferred embodiment, the p-type polymer is a mixture of PEDOT and PEDOT-OH. In a preferred embodiment, the molar ratio of PEDOT to PEDOT-OH is 1:1. The incorporation of hydroxyl groups into the polymer structure optimizes the energy levels of the bioanode-biocathode (i.e. maximizing the OCP) and provides functional groups for further anchoring enzymes on the biocathode (i.e. acting as anchor groups to promote the extent of functionalization).

Optionally, an oxygen reductase can be incorporated in the biofuel cell as a catalyst for oxygen reduction reaction (ORR). In some embodiments, the oxygen reductase is laccase or bilirubin oxidase.

C. Electrolyte Solution

The electrolyte solution is a solution that contains ions, atoms, or molecules that have lost or gained electrons, and is electrically conductive. Electrolyte solutions include but are not limited to buffers such as phosphate buffer solution (PBS), salt water, MES buffer, Bis-Tris buffer, ADA, ACES, PIPES, MOPSO, Bis-Tris propane, BES, MOPS, TES, HEPES, DIPSO, MOBS, TAPSO, Trizma, HEPPSO, POPSO, TEA, EPPS, Tricine, Gly-gly, Bicine, HEPBS, TAPS, AMPD, TABS, AMPSO, CHES, CAPSO, AMP, CAPS, CABS, or a combination thereof; biological fluids such as whole blood, serum, urine, saliva, sweat; and a combination of buffers and biological fluids. The electrolyte solution is in electrical communication with the bioanode and the biocathode. The electrolyte solution has a pH between 3 and 8.5. In the most preferred embodiment, the electrolyte solution has a pH of 7.4.

The electrolyte solution contains one or more metabolites that react with the enzymes at the bioanode to produce electrons. The metabolites are consumed in the oxidation reaction with the enzymes at the bioanode. Exemplary metabolites are glucose, glucose-1, D-glucose, L-glucose, glucose-6-phosphate, ammonia, methanol, ethanol, propanol, isobutanol, butanol and isopropanol, allyl alcohols, aryl alcohols, glycerol, cholesterol, propanediol, mannitol, glucoronate, aldehyde, carbohydrates, lactate, lactate-6-phosphate, D-lactate, L-lactate, fructose, galactose-1, galactose, aldose, sorbose, mannose, glycerate, coenzyme A, acetyl Co-A, malate, isocitrate, formaldehyde, acetaldehyde, acetate, citrate, L-gluconate, beta-hydroxysteroid, alpha-hydroxysteroid, lactaldehyde, testosterone, gluconate, fatty acids, lipids, phosphoglycerate, retinal, estradiol, cyclopentanol, hexadecanol, long-chain alcohols, coniferyl-alcohol, cinnamyl-alcohol, formate, long-chain aldehydes, pyruvate, butanal, acryl-CoA, steroids, amino acids, favin, NADH, NADH2, NADPH, NADPH2, and hydrogen. In a preferred embodiment, the metabolite is glucose, glucose-1, D-glucose, L-glucose, or glucose-6-phosphate. In the most preferred embodiment, the metabolite is glucose.

D. Membrane

Optionally, the EFC further includes a membrane that separates the bioanode and biocathode (see, for example, FIG. 24A). Any materials described above for the coating can be used for the membrane, such as Nation®. The membrane divides the EFC into two compartments such that the anodic reaction and cathodic reaction are carried out separately in each compartment.

For example, one or more metabolite is added into the electrolyte in the first compartment containing the bioanode, where the oxidation of the one or more metabolites releases electrons; the electrons are directed through an electrical connection to the biocathode in the second compartment that is separated from the first compartment by the membrane, where an oxidant is reduced to water in the second compartment.

In some embodiments, the EFC is membrane-free. In some embodiments, the membrane is absent from the EFC, and the bioanode includes a coating that encapsulate the polymeric materials and the enzymes on the surface of the bioanode.

### III. Methods of Making the Enzyme-Based Biofuel Cell

Enzyme-based biofuel cells disclosed herein contain a bioanode, a biocathode, and an electrolyte solution. The bioanode and biocathode are connected electronically. The electrolyte solution containing metabolites is in electrical communication with the bioanode and the biocathode. In the most preferred embodiments, the enzyme-based biofuel cell has a configuration as shown in FIG. 10.

The bioanode contains a conductive substrate, one or more n-type polymeric materials, and one or more enzymes, and optionally a coating. The biocathode contains a conductive substrate and one or more p-type polymeric materials. The above identified elements of bioanode or biocathode are adjacent to one another; meaning they are physically or chemically connected by appropriate means. In one embodiment, the components are physically and chemically connected by placement into a solution with an electrical connection between them. In a preferred embodiment, the components are physically connected by coating such as by spin-coating, drop-casting, printing, or electropolymerization, or otherwise deposing the individual components on the conductive substrate. In one embodiment, electropolymerization may be performed in a solution containing one or more monomers of the corresponding polymers. In another embodiment, polymerization may be performed on a surface modified with monomers via potential triggering or stimulus such as UV light or temperature. The components can be deposited separately, e.g. in layers, or they can be integrated into one deposition layer.

In a preferred embodiment, a 175 μm thick Kapton (polyimide) film is used as a conductive substrate. It is cut with a laser into a specific circular geometry (0.65 mm in diameter). A 10/100 nm thick Cr/Au layer is then sputtered on the top of the substrates. To remove any contaminants, the electrodes are cleaned in acetone and sonicated between 5 and 30 minutes, followed by a rinsing and soaking in DI water under sonication for 30 minutes. Bioanodes are made with P90 solution in chloroform spin coated on top of the active area of the conductive substrate described above by a two steps coating (500 rpm/10 s followed by a second step at 1000 rpm/30 s). Then, a 10 mg·mL⁻¹ GOx solution in phosphate buffered saline (PBS) is drop-casted on top of the active area and left to dry under ambient conditions. For making the biocathodes, on top of the general electrode cleaning procedure described above, electrochemical cleaning of the gold electrode in sulfuric acid (5 mM) via cyclic voltammetry between −0.4 and 1.2 V for 10 cycles is performed. A solution of 10 mM of 3,4-ethylenedioxythiophene (EDOT), 10 mM of Hydrooxymethyl 3,4-ethylenedioxythiophene (OH-EDOT), and 100 mM LiClO₄ was electropolymerized in an aqueous solution (pH 6.8) using potentiostatic mode at 1 V for 10 min (potentiostat Autolab PGstat128N, MetroOhm). Afterwards, the PEDOT coated biocathode is rinsed with DI water to remove excess unreacted monomers and small molecules and then dried with N2 gas.

### IV. Methods of Using the Enzyme-Based Biofuel Cell

1. Generating Electricity

One of the various aspects of the disclosed biofuel cell is a method of generating electrical power, using the bioanode of the biofuel cell to oxidize one or more metabolites such as carbohydrates, along with a biocathode to reduce an oxidant such as oxygen or peroxide. The biofuel cell can be used for both in vitro and in vivo applications. In one embodiment, the biofuel cell can be used as an in vivo electrical energy generation device utilizing glucose as metabolite and oxygen from the blood stream as oxidant. Such in vivo electrical energy generation devices may be utilized as an implanted device or power sources for implantable devices, including, but not limited to, pacemakers, insulin pumps, sensors or an array of sensors. In another embodiment, the biofuel cell can be used as in vitro for powering portable devices and wearable electronics, including, but not limited to, glucometer, sensor or an array of sensors for continuous monitoring of metabolites, ions, pH, or temperature. For example, the biofuel cell disclosed herein can generate sufficient power to drive an organic electrochemical transistor (OECT) for monitoring of metabolites, such as glucose. For example, the disclosed biofuel cell can provide sufficient power to apply a voltage at the gate electrode of an OECT, thereby switch on and off the sensor and change the drain current, consistent with the OECT operation. Additionally or alternatively, the power generated by the biofuel cell is sufficient to bias both the gate and the channel of the OECT simultaneously. For example, the polarity of the EFC biasing the gate electrode can be reversed, thereby switching the OECT from depletion mode to accumulation mode, or vice versa. Example 10 below demonstrates a fully-integrated EFC powered OECT for glucose detection. In some embodiments, the disclosed EFC can power small scale electronics, such as LED.

In another embodiment, the method of generating electrical power contains the steps of oxidizing one or more metabolites in an electrolyte solution at a bioanode and reducing an oxidant at a biocathode, wherein (a) the electrolyte solution is in electrical communication with the bioanode and the biocathode; (b) the bioanode contains a conductive substrate, one or more n-type polymers, and one or more enzymes which can react with the metabolites; (c) the biocathode contains a conductive substrate and one or more p-type polymeric materials, and wherein the bioanode is electrically connected to the biocathode.

In a preferred embodiment, biofuel cells are fabricated using (a) GOx and n-type polymers coated conductive substrate as the bioanode, (b) p-type polymers coated conductive substrate as the biocathode, and (c) biological fluid or phosphate buffer solution containing glucose wherein the glucose is at a physiological relevant concentration. Biofuel cells employing these components have demonstrated a maximum power density as high as 2.7 μW·cm⁻² and an open circuit potential of 0.31 V for 10 μM-1 mM glucose in saliva or 1-10 mM in a buffer solution such as phosphate buffered saline. In some embodiments, the disclosed EFCs employing theses components have demonstrated an OCP of at least 0.18 V, at least 0.2 V, at least 0.23 V, or up to 0.31 V. For example, the disclosed EFCs employing the above components have demonstrated an OCP of 0.18 V, 0.2 V, and 0.23 V for 250 μM, 750 μM, and 1 mM glucose respectively, which are sufficient to drive an OECT sensor. In some embodiments, the disclosed EFCs may be used in a series. For example, three of the disclosed EFCs connected in series employing the above components have demonstrated an open circuit potential of at least 1.1 V in PBS that is sufficient to drive a LED.

In some embodiments, the EFC with a membrane generates higher power density compared with the same EFC without a membrane. For example, the power density generated from an EFC with a membrane is at least 2 times, at least 3 times, at least 4 times, at least 5 times, at least 6 times, at least 7 times, at least 8 times, at least 9 times, or up to 10 times higher than the same EFC without the membrane.

In addition, the biofuel cell shows superior stability. The enzyme of the bioanode may be replenished to increase the stability of the biofuel cell.

For example, the disclosed EFC can preserve at least 15%, at least 20%, at least 30%, at least 35%, at least 40%, or at least 45% of its original maximum power density (MPD) after at least 30 days, at least 35 days, at least 40 days, at least 45 days, or at least 50 days. In some embodiments, the disclosed EFC can preserve at least 20%, between 20% and 50% of its original MPD, or between 20% and 45% of its original MPD after 30 days. Optionally, the disclosed EFC can preserve between 10% and 40%, between 10% and 30%, between 10% and 20%, or at least 15% of its original OCP after 50 days. For example, the disclosed EFC can preserve at least 45% of its original MPD after 30 days and/or preserve at least 15% of its original MPD after 50 days. In some embodiment, the disclosed EFC can preserve at least 20% or 30% of its original MPD after 30 days.

Optionally, the disclosed EFC can preserve at least 20%, at least 30%, at least 35%, at least 40%, at least 45%, at least 50%, at least 55%, at least 60%, at least 65%, or at least 70% of its original open circuit potential (OCP) after at least 30 days, at least 35 days, at least 40 days, at least 45 days, or at least 50 days. In some embodiments, the disclosed EFC can preserve between 20% and 80% of its original OCP, between 20% and 75% of its original OCP, between 20% and 70% of its original OCP, between 20% and 60% of its OCP, between 30% and 75% of its OCP after 30 days. Optionally, the disclosed EFC can preserve between 15% and 50%, between 20% and 50%, or at least 30% of its original OCP after 50 days. For example, the disclosed EFC can preserve at least 60% of its original OCP after 30 days and/or at least 30% of its original OCP after 50 days. In some embodiments, the disclosed EFC can preserve at least 70% of its original OCP after 30 days.

In some embodiments, the disclosed EFC can preserve at least 30% or at least 40% of its original MPD and at least 70% of its original OCP after 30 days. In some embodiments, the disclosed EFC can preserve at least 15%, at least 20%, at least 30%, or at least 40% of its original MPD and at least 30% of its original OCP after 50 days.

2. Sensor

Another aspect of the disclosed biofuel cell is the use as a sensor. The biofuel cell can also operate as sensors within the body. As a self-powered sensor, the biofuel cell can spontaneously and continuously monitor chemicals such as multiple metabolites that react with the enzymes both in vivo and in vitro and power the biofuel cell itself. The power output of the biofuel cell is proportional to the metabolite concentration. This provides a continuously renewable, tailored power source for applications including but not limited to targeted drug delivery, physiological monitoring and control, such as glucose monitoring.

The disclosed compounds and methods can be further understood through the following numbered paragraphs.

1. A biofuel cell comprising:

a bioanode which comprises (a) a conductive substrate; (b) one or more n-type polymers; and (c) one or more enzymes;

a biocathode which comprises (a) a conductive substrate; (b) one or more p-type polymers; and

an electrolyte solution which comprises one or more metabolite capable of reacting with the enzyme,

wherein the bioanode is electrically connected to the biocathode, and wherein the electrolyte solution is in electrical communication with the bioanode and the biocathode.

2. The biofuel cell of paragraph 1, wherein the n-type polymer is P90.
3. The biofuel cell of paragraph 1 or paragraph 2, wherein the bioanode cell further comprises coating.
4. The biofuel cell of any one of paragraphs 1-3, where in the biocathode further comprises one or more enzymes.
5. The biofuel cell of any one of paragraphs 1-4, wherein the enzyme is selected from the group consisting of glucose oxidase, glucose dehydrogenase, alcohol dehydrogenase, aldehyde dehydrogenase, formate dehydrogenase, formaldehyde dehydrogenase, lactic dehydrogenase, lactose dehydrogenase, lactate oxidase, cholesterol oxidase, tyrosinase, and pyruvate dehydrogenase.
6. The biofuel cell of any one of paragraphs 1-5, wherein the enzyme is glucose oxidase.
7. The biofuel cell of any one of paragraphs 1-6, wherein the p-type polymer is a mixture of PEDOT:PEDOT-OH.
8. The biofuel cell of paragraph 7, wherein the molar ratio of PEDOT to PEDOT-OH is 1.
9. The biofuel cell of any one of paragraphs 1-8, wherein the biocathode is capable of reducing an oxidant in the presence of electrons to form water.
10. The biofuel cell of paragraph 9, wherein the oxidant is oxygen.
11. The biofuel cell of paragraph 10, wherein the oxygen is at ambient concentration.
12. The biofuel cell of any one of paragraphs 4-11, wherein one or more enzymes are oxygen reductase.
13. The biofuel cell of paragraph 12, wherein the oxygen reductase is laccase or bilirubin oxidase.
14. The biofuel cell of any one of paragraphs 1-13, wherein the metabolite is selected from the group consisting of glucose, glucose-1, D-glucose, L-glucose, glucose-6-phosphate, ammonia, methanol, ethanol, propanol, isobutanol, butanol and isopropanol, allyl alcohols, aryl alcohols, glycerol, cholesterol, propanediol, mannitol, glucoronate, aldehyde, carbohydrates, lactate, lactate-6-phosphate, D-lactate, L-lactate, fructose, galactose-1, galactose, aldose, sorbose, mannose, glycerate, coenzyme A, acetyl Co-A, malate, isocitrate, formaldehyde, acetaldehyde, acetate, citrate, L-gluconate, beta-hydroxysteroid, alpha-hydroxysteroid, lactaldehyde, testosterone, gluconate, fatty acids, lipids, phosphoglycerate, retinal, estradiol, cyclopentanol, hexadecanol, long-chain alcohols, coniferyl-alcohol, cinnamyl-alcohol, formate, long-chain aldehydes, pyruvate, butanal, acryl-CoA, steroids, amino acids, favin, NADH, NADH2, NADPH, NADPH2, and hydrogen.
15. The biofuel cell of any one of paragraphs 1-14, wherein the metabolite is glucose, glucose-1, D-glucose, L-glucose, or glucose-6-phosphate.
16. The biofuel cell of any one of paragraphs 1-15, wherein the metabolite is glucose.
17. The biofuel cell of paragraph 16, wherein the glucose is at a physiological relevant concentration.
18. The biofuel cell of any one of paragraphs 1-17, wherein the electrolyte solution is a buffer, a biological fluid, or a combination thereof.
19. The biofuel cell of any one of paragraphs 1-18, wherein the electrolyte solution is at a pH between 3 and 8.5.
20. The biofuel cell of any one of paragraphs 1-19, wherein the electrolyte solution is at pH 7.4.
21. The biofuel cell of any one of paragraphs 1-20, wherein the reaction of the metabolite and the enzyme produces electrons which are transferred to the polymers of the bioanode.
22. The biofuel cell of any one of paragraphs 1-21 further comprising a membrane.
23. The biofuel cell of any one of paragraphs 1-22, wherein the biofuel cell preserves at least 15%, at least 20%, at least 30%, at least 35%, at least 40%, or at least 45% of its original maximum power density (MPD) after at least 30 days, at least 35 days, at least 40 days, at least 45 days, or at least 50 days.
24. The biofuel cell of any one of paragraphs 1-23, wherein the biofuel cell preserves at least 20%, at least 30%, at least 35%, at least 40%, at least 45%, at least 50%, at least 55%, at least 60%, at least 65%, or at least 70% of its original open circuit potential (OCP) after at least 30 days, at least 35 days, at least 40 days, at least 45 days, or at least 50 days.
25. The biofuel cell of any one of paragraphs 1-24, wherein the power output of the biofuel cell is proportional to the metabolite concentration.
26. The biofuel cell of any one of paragraphs 1-25, wherein the biofuel cell is utilized for powering portable devices and wearable electronics.
27. The biofuel cell of any one of paragraphs 1-25, wherein the biofuel cell is utilized for powering implantable devices
28. The biofuel cell of any one of paragraphs 1-25, wherein the biofuel cell is utilized as an implanted device.
29. The biofuel cell of any one of paragraphs 1-25, wherein the biofuel cell is utilized as an energy source to power a sensor for monitoring metabolites, ions, pH, or temperature.
30. The biofuel cell of any one of paragraphs 1-25, wherein the biofuel cell is utilized as an energy source to power an array of sensors for monitoring metabolites, ions, pH, or temperature.
31. The biofuel cell of any one of paragraphs 1-25, wherein the biofuel cell is utilized as a self-powered multi-analyte sensor.
32. A method of generating electrical power contains the steps of oxidizing one or more metabolites in an electrolyte solution at a bioanode and reducing an oxidant at a biocathode, wherein (a) the electrolyte solution is in electrical communication with the bioanode and the biocathode; (b) the bioanode contains a conductive substrate, one or more n-type polymers, and one or more enzymes which can react with the metabolites; (c) the biocathode contains a conductive substrate and one or more p-type polymeric materials, and wherein the bioanode is electrically connected to the biocathode.

The present invention will be further understood by reference to the following non-limiting examples.

### EXAMPLES

It is demonstrated for the first time the use of the n-type semiconducting polymer as an anode material in an electron mediator-free and optionally membraneless glucose-oxygen fuel cell configuration as an electrochemical energy powering source. This simple and functionalization-free fuel cell displays superior stability.

### Example 1. Glucose Oxidation on P-90/GOx is not Oxygen-Mediated

Materials and Methods

All characterizations were performed in PBS (pH 7.4). The cyclic voltammograms of the films were recorded using a potentiostat-galvanostat (Autolab, PGSTAT128N, MetroOhm) with an Ag/AgCl reference electrode (ALS co. Ltd.) and a Pt counter electrode (RE-1B, ALS co. Ltd.) in N2 or 02 saturated environments (e.g. in PBS and air) as well as in the absence or presence of glucose. The working electrode was an electropolymerized p(EDOT-co-EDOTOH) film or a P-90 film cast on top of an Au coated substrate. As for the investigation of the 02 reduction capability of the biofuel cell cathode p(EDOT-co-EDOTOH), a rotating disk electrode system (RDE710 Rotating Electrode, Gamry Instruments) was used. The film was electropolymerized on the glassy carbon electrode following the same procedure explained above. The RDE system was coupled to a channel MultiEmStat3+ (Palmsens) potentiostat and relevant voltammograms were obtained by varying the electrode rotation rate and the potential applied at a scan rate of 5 mV·s⁻¹. All experiments were performed in PBS using a Pt wire as the counter electrode and Ag/AgCl as the reference electrode.

Results

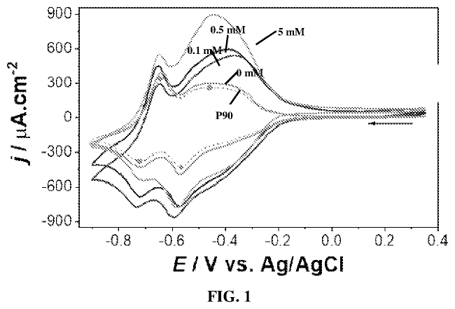

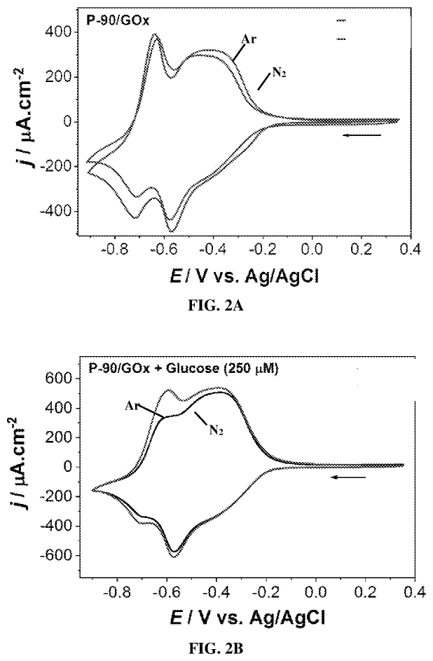

The electrocatalysis in the P-90/GOx system can be probed using cyclic voltammetry (CV). The CV curve of the P-90 film displays two quasi-reversible redox couples that are located at −0.57 V/−0.44 V and −0.73 V/−0.65 V (reduction/oxidation), characteristic of the NDI backbone (FIG. 1) (Trefz, et al., The Journal of Physical Chemistry C 2015, 119 (40), 22760-22771). After casting GOx on P-90, an enhancement was shown in both the anodic and cathodic currents with no apparent shifts in peak potentials (FIG. 1). Following the addition of glucose in the solution, the CV curve of this film undergoes significant changes involving an increase in the amplitudes of all redox peaks. With more glucose in the system, the oxidation current increases further accompanied by a decrease in the reduction current. The enzymatic reaction has the same effect on the CV curve of P-90/GOx film when recorded in air-equilibrated solutions as well as in 02-free solutions (FIGS. 2A-2D), evidencing that the oxidation of glucose in this system is not O₂-mediated (Wooten, et al., Analytical Chemistry 2014, 86 (1), 752-757).

### Example 2. P-90 Increases Adsorption of Enzymes on the Polymer Film

Materials and Methods

QCM-D measurements was conducted using a Q-sense analyzer (QE401, Biolin Scientific AB, Sweden) with Cr/Au coated quartz crystals before (used as reference) and after coating with the polymer films. After stabilizing the film in the buffer solution (PBS), the GOx solution (10 mg·mL⁻¹) was introduced into the chamber. The frequency (Δf) and dissipation (ΔD) signals were then recorded until stabilized, followed by a PBS rinsing step to allow loosely bound proteins to desorb. The measured shifts in the frequency of the sensors were converted into changes in mass (Δm) using the Sauerbrey equation:

$$Δ m = \frac{- 17.7}{n + Δ f} \qquad ( 1 )$$

where n is the number of the selected overtone for the quantification of the mass and −17.7 is a constant determined on the resonant frequency, active area, density and shear modulus of the crystal (Savva, et al., Journal of Materials Chemistry C 2018).

Results

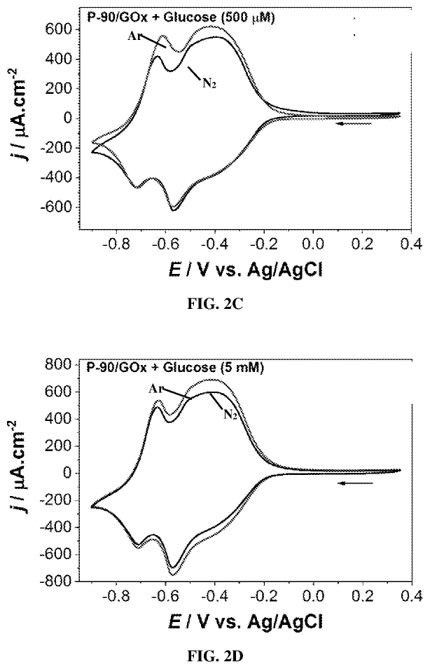

Using quartz crystal microbalance with dissipation monitoring (QCM-D) studies, the amount of enzyme adsorbed on the film was quantified. The enzyme-induced changes in the oscillation frequency and dissipation of a QCM-D crystal coated with P-90, as well as with its ethylene glycol free analogue, P-0 (the ratio of glycol:alkyl side chains is 0:100) were measured (FIG. 3). When the P-90 film is incubated with GOx, the enzyme adsorbs on the polymer surface without prior surface treatment. A substantial amount of GOx remains on top of P-90 after being rinsed with PBS (ca. 110 ng·cm⁻², i.e., 7% of the initial amount is desorbed upon rinsing). On the contrary, most of the enzyme initially adsorbed on the P-0 surface is washed away (ca. 50 ng·cm⁻² remaining, i.e., 50% of the initial amount is desorbed upon rinsing). The presence of ethylene glycol content of the NDI-T2 film is important for establishing interactions between the protein and the polymer, enabling the functionalization of P-90 with GOx. A similar phenomenon was observed when investigating the interactions of synthetic zwitterionic lipid vesicles with a library of P-90 analogs including the P-0 (Zhang, et al., ACS Applied Bio Materials 2018, 1 (5), 1348-1354)). Liposomes favored the ethylene glycol-rich regions on a given film surface and did not adhere on the P-0 film which has limited wettability.

### Example 3. The Enzyme and P-90 have an Interface, Leading to Efficient Electronic Communication

Materials and Methods

P-90 film was coated on an ITO substrate following the a two steps spin coating procedure (350 rpm/10 s followed by a second step at 1000 rpm/30 s). An Ocean Optics QE Pro Scientific grade spectrometer (185-1050 nm) was used to record the UV-VIS-NIR spectra of the films. For the spectroelectrochemistry measurements, a Keithley 2606A source measure unit was coupled to the sample holder which contains the film submerged in the electrolyte. When required, a bias was applied between an Ag/AgCl electrode and the P-90 film addressed as the working electrode, in the absence or presence of the enzymatic reaction.

For the in situ Raman spectroelectrochemical investigation, P-90 films coated on Au substrates were exposed to a drop of PBS into which an Ag/AgCl electrode was immersed. The electrochemical area (1 cm²) was defined as a square aperture in a Parafilm medium where 5 μL of the solution (PBS, with or without glucose) was placed. The bias was then applied using a Keithley 2600B source meter. The near-resonance Raman spectra were measured using a Witec alpha Raman spectrometer in backscattering configuration with a linearly polarized excitation of He—Ne laser of wavelength 632.8 nm, and a power level <500 μW to avoid photo-thermal effects. The dispersion gratings used, 600 g·mm⁻¹, allowed to collect a spectral range up to 2700 cm⁻¹, covering the spectral area of interest completely. A Zeiss 63x, NA=1, water immersion objective focused on the polymer surface was used to excite the sample and collect the Raman signal. For each condition, the sample was mapped with 10 points for 2 sec to average out local statistical fluctuation, thus defining the representative spectrum. The Raman spectra presented here were obtained by removing the bias-dependent baseline, well described by a 4^{th} order polynome, and after the normalization to the [symbol] -mode peak intensity of the bithiophene units (1457 cm⁻¹). The baseline treatment allowed tracking the bias-triggered evolution of the polaronic excitation in the copolymer backbone responsible for strong light absorption, emission, and Raman scattering. Using this procedure, the relative intensities and shifts of the main peaks recorded for different samples and at various biasing conditions were compared in the absence and presence of enzymatic reaction.

Results

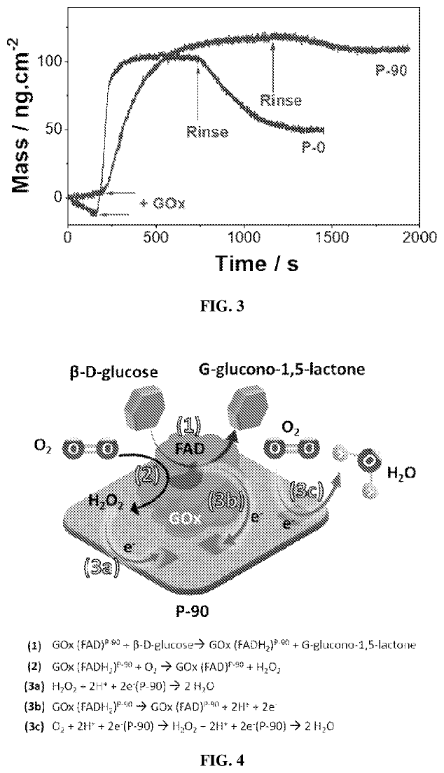

The P-90 film accepts electrons of the enzymatic reaction and then transports them along its backbone on the condition that the FADs are in the proximity (see possible reactions in the P-90/GOx system summarized in FIG. 4). Further evidence for this mechanism comes from the optical absorption spectrum of P-90, which displays distinct features associated with its doping state (Savva, et al., Journal of Materials Chemistry C 2018).

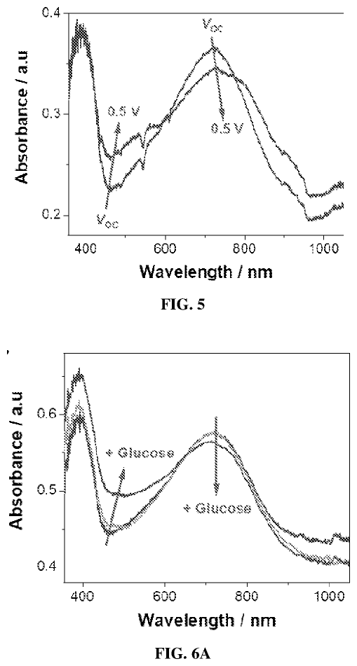

FIG. 5 shows the voltage-induced changes in the UV-VIS absorbance spectrum of a P-90 film as it undergoes from a neutral to an electrochemically reduced state in PBS (i.e., doped by cations). When a doping potential is applied, the intensity of the low energy absorption feature decreases while a new peak around 450 nm arises (Giovannitti, et al., Chemistry of Materials 2018, 30 (9), 2945-2953; Giovannitti, et al., Nature Communications 2016, 7, 13066).

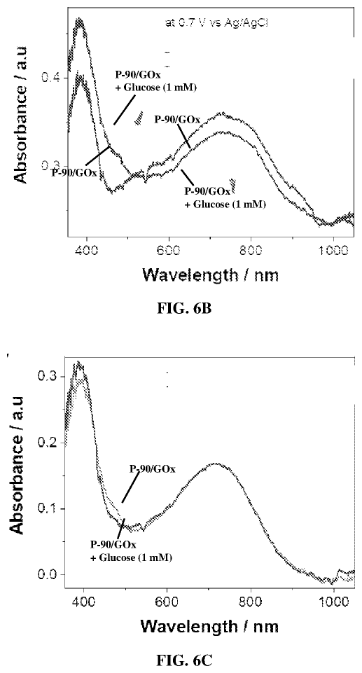

FIGS. 6A and 6B show that similar changes occur for the P-90/GOx system yet these are triggered by the addition of glucose in the solution. H₂O₂, on the other hand, has no such effect on the spectrum of P-90 (FIG. 6C). The enzymatic reaction thus perturbs the electronic structure of the polymer, emulating the doping voltage.

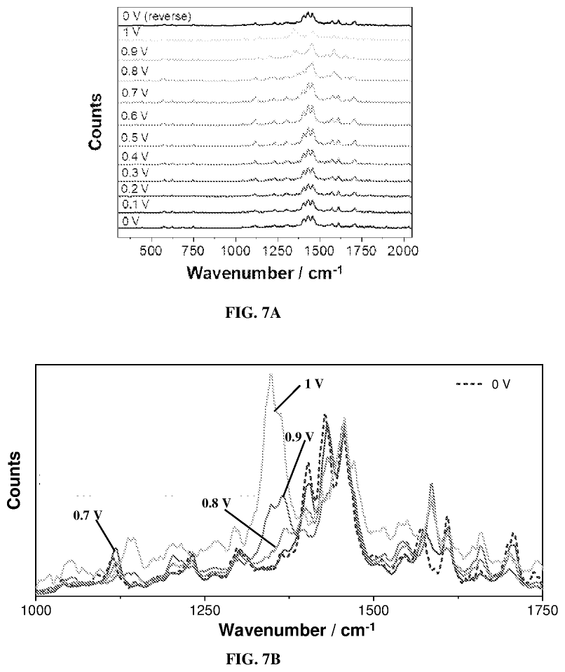

To track these events at the molecular level, the Raman spectra of P-90 during electrochemical doping and the course of enzymatic reactions was recorded. FIG. 7A displays the evolution of the Raman spectrum of a P-90 film subject to increasing doping potentials. In the P-90 spectrum, the region between 1100 and 1800 cm⁻¹ refers to the resonant region of carbon bonds in the molecule (i.e., backbone), while the low energy region (<1100 cm⁻¹) are associated with the side-chains. The three main peaks in the former region are attributed to the collective C═C—C bonds stretching/shrinking confined on the T2 unit (1457 cm⁻¹) as well as on the NDI unit (1407 cm⁻¹), and to the delocalized vibrations of the NDI-T2 monomer (1431 cm⁻¹) (FIG. 7C) (Giussani, et al., Macromolecules 2013, 46 (7), 2658-2670). These modes, called [symbol] -modes, represent the vibrational trajectory that best favors the oscillations of the C═C—C bonds-length alternation, and as such, they are the most sensitive to the π-electron perturbations (Navarrete, et al., The Journal of Chemical Physics 1991, 94 (2), 965-970). In other words, vibrations have only a partial [symbol] character, so the intensity of such modes is proportional to their [symbol] fraction, which is responsible also for its dispersion character. Moreover, the peaks at 1709 cm⁻¹, 1612 cm⁻¹, and 1574 cm⁻¹ are attributed to symmetric stretching vibrations of C═O, C═C, and C═N of the NDI unit, respectively. Finally, other minor vibrations at 1118, 1232, and 1301 cm⁻¹ are related to CH and CH2 modes.

When the film is biased at 0.7 V, its spectral profile shows peaks that have changed in intensity and position and the extent of these changes increases with the doping voltage, showing that structural rearrangements occur concurrently with the localization of the electrons on the backbone (FIG. 7B). The switch in the electrochemical state affects mainly the [symbol] -mode of the NDI unit: the characteristic peak at 1407 cm⁻¹ reduces in intensity as the doping voltage increases up to 1 V. Meanwhile, two lower frequency peaks located at 1347 and 1364 cm⁻¹ gain in intensity and dominate the spectrum. These peaks are to be attributed to the dopant cations that generate strongly localized defects, transforming the T2 unit structure from an aromatic to a more quinoid form. Finally, as the voltage is reverted to 0 V, the spectrum recovers its original shape (FIG. 7A).

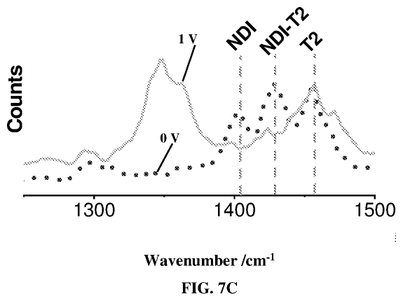

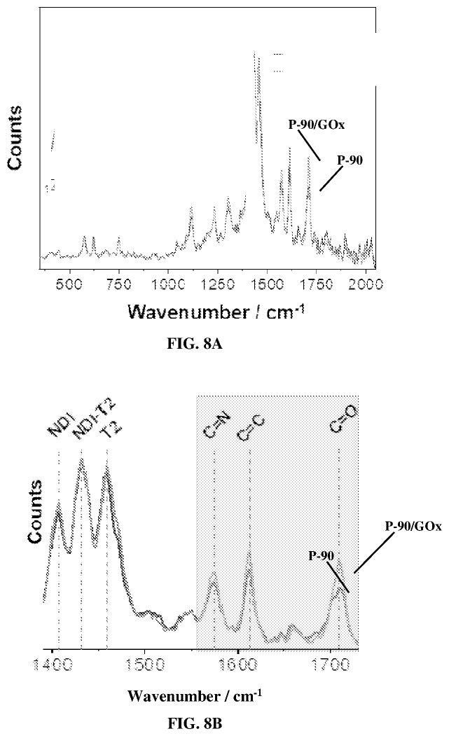

Upon adsorption of the enzyme on the P-90 film, a minor increase in the intensity of the C═O, C═C, and C═N peaks of NDI unit was observed, while the [symbol] -modes remain unaffected (FIGS. 8A and 8B). No new peaks was observed, due to the non-resonant (limited) Raman efficiency of the protein. Raman signals are sensitive to changes in the polarizability of the molecule. The polarizability change induced by the enzyme is lower than that induced by electrochemical doping (compare FIGS. 7B and 7C with FIGS. 8A and 8B), showing that there is a partial overlap of the electron clouds of the two molecules due to their proximity, affecting the electronic charge distribution of the backbone. The enzymatic reaction of glucose with GOx causes significant shifts and intensity changes of the Raman peaks (FIGS. 9A and 9B). As glucose is introduced to the solution, changes similar to those observed upon electrochemical doping were observed (although the voltage is kept constant at 0.7 V): [symbol] -mode on the NDI loses its intensity while the neighboring lower energy peaks become prominent. As GOx catalyzes glucose, P-90 gets doped as if it is electrochemically addressed.

These results show that the active sites of the enzyme and the copolymer have an interface which leads to an efficient electronic communication. At this bio-electronic interface, analyte oxidation increases the conductivity of the polymer by donating new electrons to the backbone, and this process proceeds without the aid of an external electron mediator.

### Example 4. Enzymatic Biofuel Cell Structure

Materials and Methods

Electrodes Fabrication

A 175 μm thick flexible Kapton (polyimide) films as substrate. Kapton was cut with a laser into a specific circular geometry (0.65 mm in diameter) and subsequently washed in acetone/IPA and deionized water baths under sonication for 30 min. A 10/100 nm thick Cr/Au layer was then sputtered on the top of the substrates. To remove any contaminants, the electrodes were cleaned in acetone and sonicated for 30 minutes, followed by a rinsing and soaking in DI water under sonication for 30 minutes.

Bioanode: n-type semiconducting polymer (P90) solution (10-15 μL aliquots) in chloroform was spin-coated on top of the active area of the Au coated flexible polyimide substrate (0.33 cm²) by a two steps coating (350 rpm/10 s followed by a second step at 1000 rpm/30 s). Upon natural drying of the film, GOx solution in phosphate-buffered saline (PBS) (10 mg mL⁻¹) was drop-casted on top of the electrode (i.e., immobilization of GOx through enzyme adsorption) and left to dry under ambient conditions for a minimum of 30 minutes.

Biocathode: On top of the general electrode cleaning procedure, electrochemical cleaning of the gold electrode in sulfuric acid (5 mM) via cyclic voltammetry between −0.4 and 1.2 V for 10 cycles was performed. A solution of 10 mM of 3,4-ethylenedioxythiophene (EDOT), 10 mM of Hydrooxymethyl 3,4-ethylenedioxythiophene (OH-EDOT) and 100 mM LiClO₄ was electropolymerized in an aqueous solution (pH 6.8) using potentiostatic mode at 1 V for 10 min (potentiostat Autolab PGstat128N, MetroOhm). Afterwards, the PEDOT coated electrode was rinsed with DI water to remove excess unreacted monomers and small molecules and then dried with N₂ gas.

Electrochemical Cell Assembly

Glass vials (Ossila, C20052) were used for the membrane-free cells where the inter-electrode gap distance was ˜0.5 cm. For the membrane cell, a cationic exchange membrane separated the anode and the cathode at a distance of ˜1.5 cm (Nafion 117, Sigma Aldrich) to maintain electroneutrality.

The custom-built EFC was made from poly(methyl methacrylate) (PMMA) and could accommodate 2 mL of the solution on each side.

Scanning Electron Microscopy (SEM) Measurements

SEM images were obtained using Nova Nano SEM. P-90 films were deposited on glass coverslips and coated with a 5 nm of iridium before imaging. For the wet conditions, the films were immersed in deionized water overnight to ensure that they swell. The samples were then frozen using liquid N2 and sublimated inside the cryo-SEM chamber.

Calculation of Electron Transfer Number

The number of electrons involved in the ORR is extracted from the slope of each curve (B) and plotted for a specific potential of the voltammogram according to the following equation (Mao, et al., Energy & Environmental Science 2014, 7 (2), 609-616):

$$n = \frac{B}{0.62 {FC}_{o} {D}_{o}^{2 / 3} {v}^{- 1 / 6}}$$

where n is the number of electrons, B is the slope of each curve, F is the Faraday constant (96485 C·mol⁻¹), Cₒ is the saturated concentration of O₂ in the electrolyte (1.2×10⁶ mol·cm⁻³), Dₒ is the O₂ diffusion coefficient (1.9×10⁻⁵ cm²·s⁻¹)²⁸ and ν is the kinematic viscosity of the electrolyte (PBS).

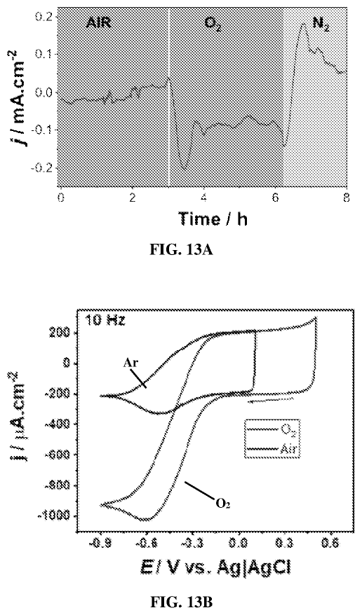

According to FIG. 13C, at low negative potentials (−0.375 V) the system follows a 4 e⁻ reduction of oxygen to water. As the potential increases in magnitude, the number of electrons involved in the ORR decreases to 2, indicative of an indirect ORR pathway proceeding with H₂O₂ formation.

Results

A typical EFC includes two electrodes, an anode, and a cathode, connected via an external load (Aghahosseini, et al., Nanochemistry Research 2016, 1 (2), 183-204). Since P-90 film has electronic coupling with the enzyme, i.e., its conductance is enhanced upon the catalytic reaction of GOx with glucose, P-90/GOx film is employed as an anode for an enzymatic biofuel cell (EFC). FIG. 10 depicts the schematic of a membrane-free EFC configuration where flexible Au-coated polyimide is used as substrate that carries cathode or anode materials, and the electrolyte is the PBS or saliva solution containing glucose. No electron mediators are integrated in the cathode or the anode while the cathode is not relying on an enzyme. Given the simple assembly of the electrodes, the possibilities of both incorporating and omitting a membrane (i.e., Nafion) in the cell to separate the anodic and cathodic compartments were explored.

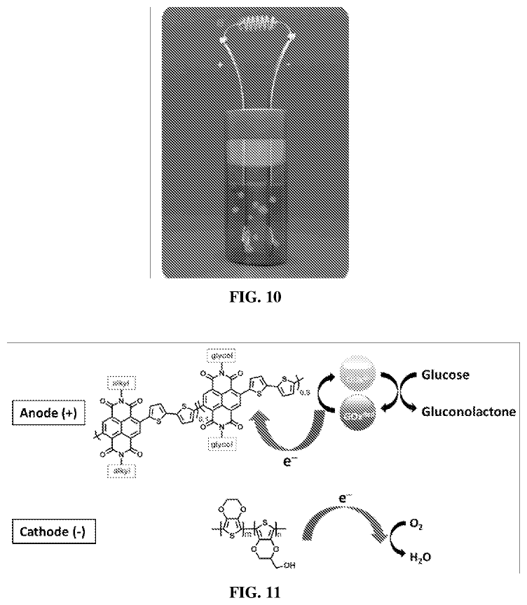

Upon introduction of glucose into PBS, the former is oxidized to gluconolactone by GOx, producing electrons that are transferred to the P-90 anode (FIG. 11). These electrons travel through the external circuit to the cathode which reduces dioxygen to water so that the circuit generates power from glucose and 02 (Luz, et al., ChemElectroChem 2014, 1 (11), 1751-1777).

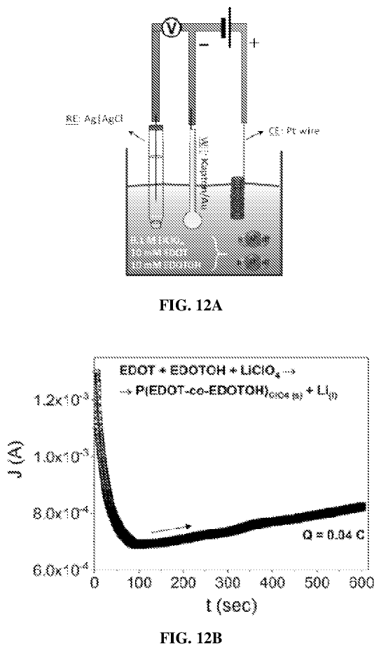

The cathode is p(EDOT-co-EDOTOH), a p-type copolymer that was electropolymerized on a gold-coated surface with an area identical to the anode (FIGS. 12A and 12B). This material was selected as the cathode due to the ability of PEDOT derivatives to reduce O₂, as well as the simplicity and low cost of fabrication (Mitraka, et al., Journal of Materials Chemistry A 2017, 5 (9), 4404-4412; Singh, et al., The Journal of Physical Chemistry C 2017, 121 (22), 12270-12277; Winther-Jensen, et al., Science 2008, 321 (5889), 671-674; Mitraka, et al., Advanced Sustainable Systems 2019, 3 (2), 1800110). The p(EDOT-co-EDOTOH) exhibits 02 reduction reaction (ORR) as evidenced by the enhancement of the reduction current in O₂ saturated environments (FIGS. 13A-13C).

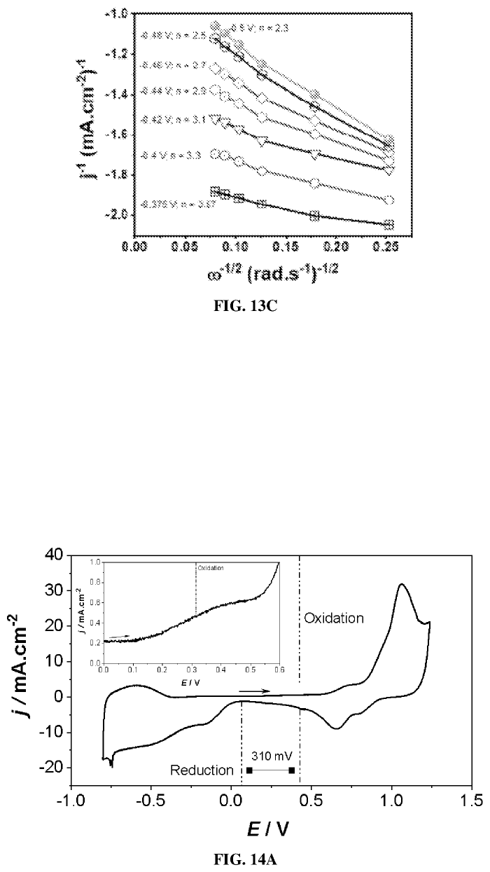

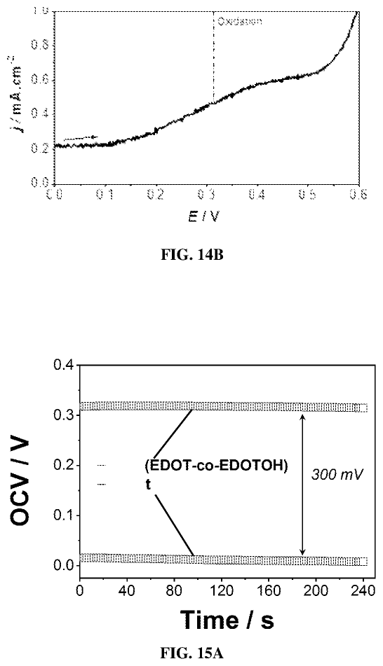

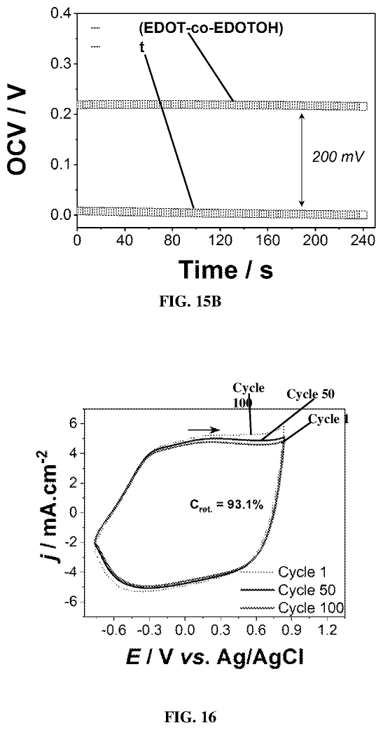

The potential difference that corresponds to the onset potentials of the glucose oxidation and O₂ reduction potentials of the EFC is evaluated by CV experiments (Ecell=˜0.5 V) and shown in FIGS. 14A and 14B. The polymeric cathode leads to a higher open circuit voltage compared to Pt, a common cathode of biofuel cells (FIGS. 15A and 15B), and exhibits good stability against continuous cycling with a capacitance retention of 93% upon 100 CV cycles (FIG. 16).

### Example 5. The n-Type Polymer Allows Fast Heterogeneous Electron Transfer

Materials and Methods

Electrochemical Characterization

The half-cells and biofuel cells were electrochemically characterized using a MultiEmStat3+(Palmsens) potentiostat. For half-cell characterization, cyclic voltammograms at ambient temperature were recorded in a three-electrode set up using an Ag/AgCl reference electrode and a Pt foil counter electrode. P-90 film and electropolymerized p(EDOT-co-EDOTOH) were coated on Au sputtered substrates for anode and cathode characterization, respectively. For the measurements performed under inert atmosphere, the system was degassed in a closed chamber for at least 15 minutes in N₂ prior to measurement.

Calculation of k_{ET}

The anodic and cathodic peak-to-peak separation (ΔFₚₚ=Eₚ−E_{1/2}) at high scan rates (i.e., ν=100 mVs⁻¹) should be linear to the logarithm of the scan rate:³⁰

$${E}_{PC} = {E}^{o} - \frac{R \text{?} T}{α \text{?} n \text{?} F} ln ( \frac{α \text{?} n \text{?} F}{R \text{?} T \text{?} {k}_{ET}} * v )$$

$${E}_{PA} = {E}^{o} + \frac{R \text{?} T}{( 1 - α ) \text{?} n \text{?} F} ln ( \frac{( ⊥ - α ) \text{?} n \text{?} \text{?}}{R \text{?} T \text{?} {k}_{ET}} * v )$$

$$\text{?} \text{indicates text missing or illegible when filed}$$

where E^{o} is the formal potential (E_{Ox}−E_{Red}), n is the number of transferred electrons, α is the electron transfer coefficient (related to the symmetry of the redox reaction), and ν is the scan rate. From the slope of the curves, the a values were assigned equal to 0.5, demonstrating a symmetrical redox process, while n was ca. 1.72, close to the two electron reaction involved in the glucose oxidation. k_{ET} can then be extracted as:

$$log ( {k}_{ET} ) = α * log ( 1 - α ) + ( 1 - α ) * log ( α ) - log ( \frac{R * T}{* n * F * v} ) - α ( 1 - α ) * \frac{{Δ E}_{P} * n * F}{2.3 * R * T}$$

The k_{ET} of the P-90/GOx system is ca. 8.11 s⁻¹ at 400 mV·s⁻¹.

Results

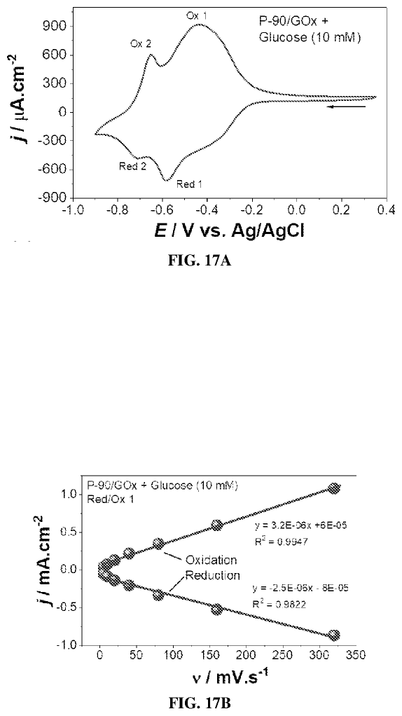

A scan rate dependence study of the current of the bioanode generated in the presence of glucose reveals a linear relationship between the current and the scan rate, indicative of surface-controlled processes (FIGS. 17A-17D) (Eckermann, et al., Coordination Chemistry Reviews 2010, 254 (15), 1769-1802; Salimi, et al., Biosensors and Bioelectronics 2007, 22 (12), 3146-3153). Elucidating this curve, we extracted the heterogeneous electron transfer rate constant, k_{ET}, following the Laviron model (Eckermann, et al., Coordination Chemistry Reviews 2010, 254 (15), 1769-1802; Laviron, et al., Journal of Electroanalytical Chemistry and Interfacial Electrochemistry 1979, 101 (1), 19-28).

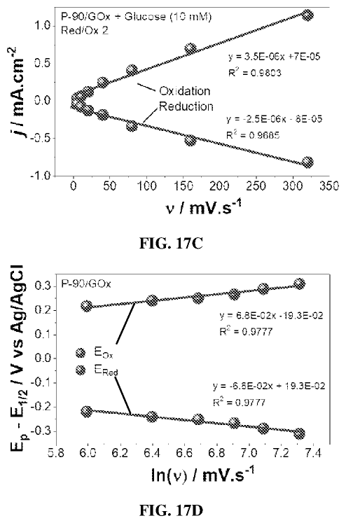

The k_{ET} of the P-90/GOx system is ca. 8.11 s⁻¹ at 400 mV·s⁻¹ (Table 1). This result demonstrates the seamless nature of the polymer/enzyme interface and the ability of the polymer to provide a suitable environment for GOx to transfer electrons. Notably, the P-90 film remains stable upon consecutive cycling (100 cycles) with a current retention greater than 97% (FIG. 18).

TABLE 1
Comparison of k_{ET} values found in this
work with reported values of other EFCs.

| Template for GOx | k_{ET}/s⁻¹ | Ref. |
|---|---|---|
| CNT/GA/POx | 0.046 | ₁₁ |
| MCF(TOA-Au NP)/ | 6.0 | ₁₂ |
| GOx | | |
| CNT/TiO₂/GOx | 3.96 | ₁₃ |
| PEI/CNT/GA/GOx | 8.6 | ₆ |
| CP/GOx | 12.1 | ₁₄ |
| Graphene/SWCNT | 0.23 | ₁₅ |
| cogel/GOx | | |
| CNT/PEI/PCA/GOx | 11.5 | ₁₆ |
| CNC/CS/GC/GOx | 6.0 | ₁₇ |
| CNT-modified/ | 1.0 | ₁₈ |
| GC/GOx | | |
| Au/P-90/GOx | 8.1 | This work |

POx: Pyranose oxidase; PEI: poly(ethylenimine); MCF: Metallic cotton fiber; CP: Carbon paper; PCA: pyrenecarboxaldehyde; CNC: Carbon nanochips; CS: Chitosan; GC: Glassy carbon electrode; TOA-Au NP: tetraoctylammonium bromide-stabilized Au nanoparticle.

The fuel cells disclosed in the references cited in Table 1, use carbon based materials (CNTS, carbon paper etc. . . . ) and relatively complicated electrodes fabrications (multilayers, enzyme immobilization, etc.). By contrast, the disclosed biofuel eliminates the need of any functionalization process used in prior system, while demonstrating impressive levels of stability.

### Example 6. The EFC Generates High Power Density

Materials and Methods

EFC Characterization

For the EFC characterization, various glucose concentrations were supplied to the biofuel cell using a peristaltic syringe pump (Ossila, L2003S1) and the open circuit voltage (OCV) of the cell was recorded throughout 30-minute intervals. Power curves were obtained by measuring the cell voltage across a variable load resistor (1 kΩ-10 MΩ). Once a resistor of fixed value was applied, each point was measured after 30 minutes of stabilization period to ensure a stable voltage output. Using Ohm's law, current and power densities were calculated using the geometrical surface area of the electrodes. For estimation of power densities at extended potentials, linear sweep voltammetry (LSV) curves (up to 1.2.V) were recorded at a scan rate of 5 mV·s⁻¹. To obtain the inflection points and corresponding power retentions, the first derivative of the LSV plot was calculated. The total volume of the solution used for each measurement was 1 mL. The operating temperature of the EFC was 25° C.

Hydrogen Peroxide Detection

For the detection of hydrogen peroxide (H₂O₂), aliquots (0.1 mL) of the EFC electrolyte were collected during its operation with a disposable syringe (Terumo). A peroxide assay kit (Sigma Aldrich) was used to determine the concentration of H₂O₂ in these aliquots. The assay utilizes the chromogenic Fe³⁺-xylenol orange reaction, in which a purple complex is formed when Fe²⁺ is oxidized to Fe³⁺ by the H₂O₂ present in the sample, generating a colorimetric result (585 nm). A spectrophotometer (Promega) was used to measure the absorbance intensity, which scales with H₂O₂ concentration.

Results

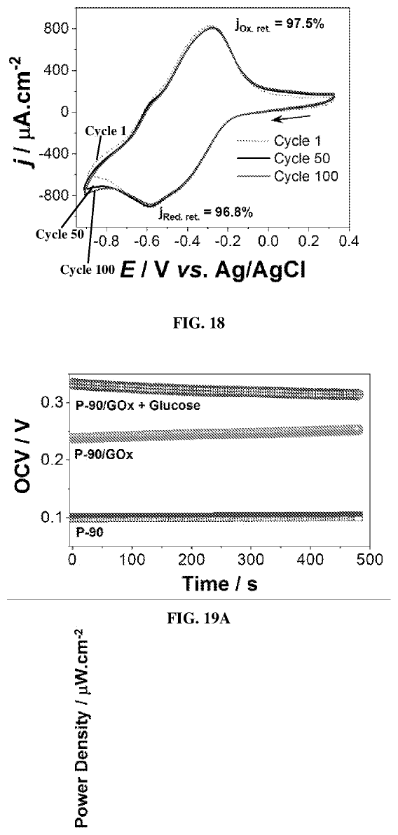

FIG. 19A portrays the evolution of OCV in the absence and presence of the enzyme and glucose. Upon enzymatic reaction with 1 mM of glucose, ca. 0.32 V was drawn from the EFC under open circuit conditions, somewhat lower than the theoretical OCV, attributed to overpotential loss originating from the semiconducting nature of the anode. The power density curves of the EFC are shown in FIGS. 19B and 19C. As glucose is introduced in the solution, a clear increase in the power generated by the EFC was observed, reaching a maximum of 2.8 μW·cm⁻² at 10 mM of glucose.

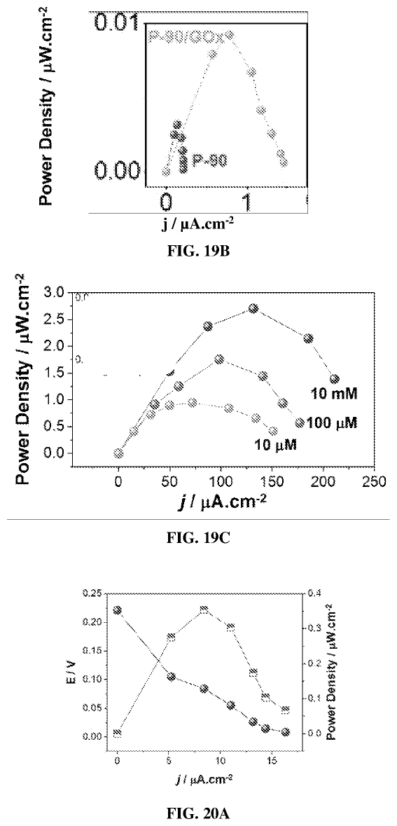

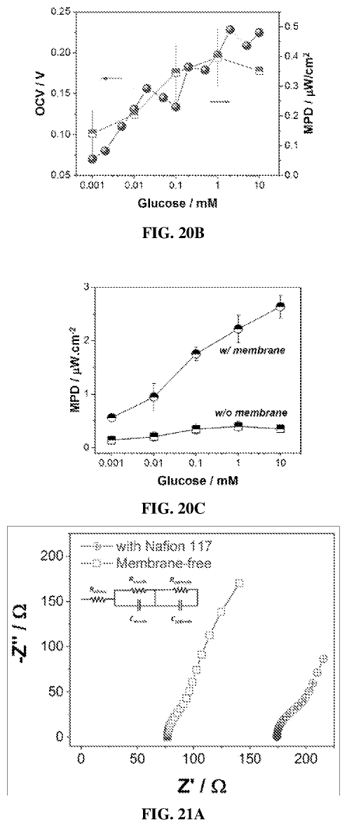

The possibility to use a membrane-free configuration was explored by removing the Nafion membrane. The cell had a lower maximum power density (MPD) than the membrane-based EFC (i.e., 0.4 μW·cm⁻² vs 2.8 μW·cm⁻² for 10 mM of glucose) (FIGS. 20A-20C) despite its reduced internal resistance (FIGS. 21A and 21B).

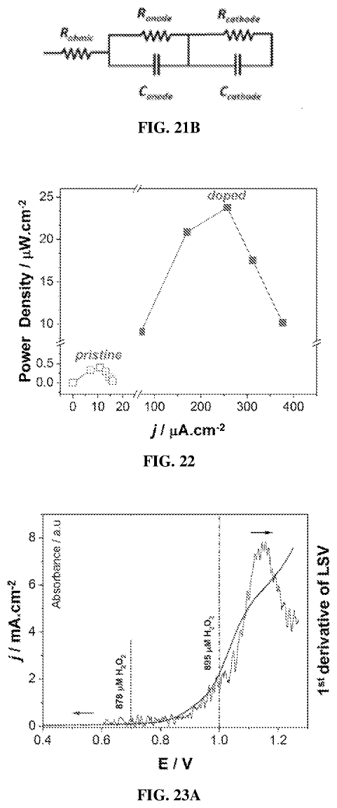

One poignant characteristic of n-type semiconducting polymer-based devices is their low electrical conductivity, which contributes significantly to the device internal resistance and hence hindering a high-power output. Indeed, the MPD increases ca. 60-fold for the biofuel cell comprising an electrochemically doped P-90 at the anode (FIG. 22). Using the semiconducting polymer at the anode in its conducting form is a simple demonstration of how the performance of this all-polymer biofuel cell can be improved. Modification of the semiconducting polymer include doping the n-type film with molecular dopants (Liu, et al., Advanced Materials 2018, 30 (7), 1704630), increasing the planarity of its backbone (Wang, et al., Advanced Materials 2018, 30 (31), 1801898), turning to polymer composites with conducting particles (Cho, et at, Advanced Materials 2015, 27 (19), 2996-3001), controlling the ordering and multi-scale assembly of the chains via processing means (Rivnay, et al., Advanced Materials 2010, 22 (39), 4359-4363), to name a few.

### Example 7. The EFC is Stable for a Month

Materials and Methods

The stability of the biofuel cells was tested by measuring the change in their OCV and the power density values over time. A syringe pump with a rate of 150 μL·s⁻¹ was used to feed the cell. The effect of enzyme replenishment as well as of enzyme encapsulation was investigated (using a Nafion 117 film) on device stability. The electrodes were stored under ambient conditions after each measurement.

Results

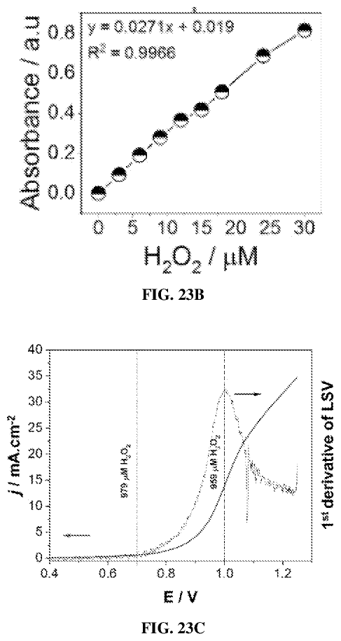

The stability of the biofuel cells was investigated by monitoring the OCV. The behavior under non-equilibrium conditions (beyond the OCV) was examined when the cell is biased at high positive voltages (>0.6 V). At this extended biasing regime, both membrane-free and membrane-based EFCs produce a high amount of H₂O₂ responsible for the large currents generated (FIGS. 23A-23C) (Miglbauer, et al., Chemical Communications 2018, 54 (84), 11873-11876). Unless it is operated at high positive voltages, the cell does not produce H₂O₂: the oxidation of glucose at the anode is not O₂-mediated (FIGS. 2A-2D) while the ORR of the cathode proceeds through the pathway that leads to water (FIGS. 13A-13C).

When the enzyme is not replenished, after 30 days of use, the OCV drops to 30% of its original value while the EFC preserves ca. 40% of the power that it produces when biased up to the H₂O₂ production regime (FIG. 24B). If the enzyme is replenished between measurements, the EFC withholds 60% and 45% of its OCV and PD, respectively. In this configuration, the PD is stable for an additional period of 20 days (50 days in total) (FIG. 24C). Having the Nafion film cast directly on top of P-90/GOx instead of using a compartment separator (FIG. 24E) improved the stability of the cell: the EFC maintains in average 45% of its OCV and 30% of its PD after 30 days. This direct encapsulation strategy aids in enzyme stabilization when intended for long term use.

The stability studies revealed that in the current configuration, the EFC stability is mainly challenged by enzyme denaturation and instability of the p-type polymer coating. Diligent choice of encapsulation materials and optimization of the cathode are effective strategies to improve the overall stability of the EFC. FIG. 24E shows that the stability is improved when the P-90/GOx film is encapsulated by Nafion coating (EFC maintains 45% of its OCV after 30 days of use). The encapsulation strategy aids in enzyme stabilization when intended for long term use.

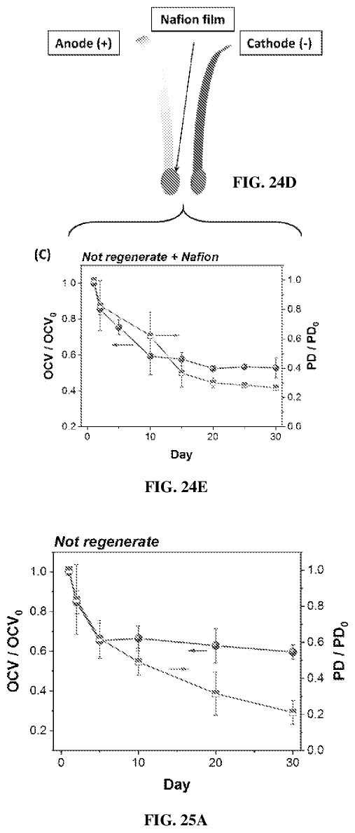

As for the membrane-free devices, when the enzyme is not replenished (FIG. 25A), the OCV drops to 53% of its original value after 30 days, while the EFC retains 20% of its initial MPD, mainly due to the denaturation of the enzyme. Upon continuous replenishing of the GOx on P-90 (FIG. 25B), superior performance is obtained, with an OCV and PD retention of 70% and 30%, respectively. The membrane-free EFCs show stability on par with the ones having a membrane, demonstrating their ability to generate the power to operate a sensor or actuator continuously.

Taken together, the polymers selected as the anodic and cathodic coatings exhibit intrinsic catalytic properties on a level competitive with the other reported systems—which have undergone exhaustive device performance optimization—while benefiting from the ease of electrode preparation (Table 2).

TABLE 2
Comparison of the performance of previously reported
glucose EFCs with this work

| Anode | Cathode | | | MPD | | |
|---|---|---|---|---|---|---|
| (Electrode | (Electrode | | OCV | μW · | | |
| support) | Support) | Solution | (V) | cm⁻²) | Stability | Ref. |
| CDH | Laccase | Citrate buffer | 0.73 | >5 | 25 h | ₁₉ |
| (spectrograp | (spectrograp | pH 4.5, | | | | |
| hic | hic | 5 mM glucose | | | | |
| graphite) | graphite) | | | | | |
| CDH | BOx | PBS buffer, | 0.62 | 3 | >6 h | ₂₀ |
| (graphite) | (graphite) | pH 7.4, | | | | |
| | | 5 mM glucose | | | | |
| | | Serum | 0.58 | 4 | <2 h | |
| GOx | Laccase | PBS buffer, | 0.23 | 31 | — | ₂₁ |
| (Au/Nafion- | (Au/Nafion- | pH 7.4, | | | | |
| PVP | PVP | 5 mM glucose | | | | |
| nanowire/CNT) | nanowire) | | | | | |
| GOx | Laccase | PBS buffer, | 0.95 | 1300 | 2.7 h | ₂₂ |
| (CNT disk) | (CNT disk) | pH 7.4, | | | | |
| | | 5 mM glucose | | | | |
| CDH | BOx | PBS buffer, | 0.66 | 3.2 | 30 h | ₂₃ |
| (Au NPs) | (Au NPs) | pH 7.4, | | | | |
| | | 5 mM glucose | | | | |
| CDH | Box | tear | 0.57 | 3.5 | >10 h | ₂₄ |
| (Au NPs/Au | (Au NPs/Au | | | | | |
| μ-wire) | □ □ wire) | | | | | |
| GOx | BOx | PBS buffer, | 0.93 | 40.8 | — | ₉ |
| (Carbon | (Carbon | pH 7.4, | | | | |
| nanodots) | nanodots) | 4 mM glucose | | | | |
| GOx | Laccase | 0.01 M PBS | — | 102 | 77% of | ₆ |
| (PEI/CNTs) | (PEI/CNTs) | buffer, pH 3, | | | MPD | |
| | | 40 mM | | | after 4 | |
| | | glucose, 100 | | | weeks | |
| | | cc · min⁻¹ O₂ | | | | |
| | | flow | | | | |
| GOx | Laccase | PBS buffer, | 0.78 | 1120 | 83% of | ₂₅ |
| (Glassy | (Glassy | pH 7.2, | | | current | |
| carbon/PAN | carbon/PAN | glucose | | | density | |
| I-CNT) | I-CNT) | concentration | | | after 15 | |
| | | not mentioned | | | days | |
| GOx | (Pt) | PBS buffer, | 0.59 | 55 | — | ₁₇ |
| (Glassy | | pH 7.2, 10 mM | | | | |
| carbon/Carb | | glucose | | | | |
| on | | | | | | |
| nanochips) | | | | | | |
| GOx | Laccase | 0.2 mM UA, | — | 180 | 89% of | ₇ |
| (PEI/CNTs) | (PEI/CNTs) | 0.9 mM AA, | | | the MPD | |
| | | 140 mM | | | after 16 | |
| | | NaCl, 5 mM | | | days | |
| | | glucose | | | | |
| GOx/Fc | BOx | PBS buffer, | 0.43 | 34.3 | 24 h | ₂₆ |
| (graphene | (graphene | pH 7.4, | | | | |
| coated carbon | coated carbon | 200 mM glucose | | | | |
| fiber cloth) | fiber cloth) | | | | | |
| FAD/GDH/ | BOx/ | PBS buffer, | 0.78 | 360 | >1.5 h | ₂₇ |
| NQ-LPEI | AnMwCNT | pH 7.4, 50 mM | | | | |
| (Toray carbon | (Toray carbon | glucose/pH | | | | |
| paper) | paper) | 5.6 for catholyte | | | | |
| GOx (P-90) | p(EDOT-co- | PBS buffer, | with | with | with | This |
| | EDOTOH) | pH 7.4, 10 mM | membrane: | membrane: | membrane: | work |
| | | glucose | 0.31 | 2.7 | 40% of | |
| | | | | | power | |
| | | | | | density | |
| | | | | | recorded at | |
| | | | | | 1 V/30% | |
| | | | | | OCV after | |
| | | | | | 50 days | |
| | | | membrane- | membrane- | without | |
| | | | free: 0.185 | free 0.4 | membrane: | |
| | | | | | 40% of | |
| | | | | | power | |
| | | | | | density | |
| | | | | | recorded at | |
| | | | | | 1 V/75% | |
| | | | | | OCV after | |
| | | | | | 30 days. | |

CDH: cellobiose dehydrogenase; BOx: bilirubin oxidase; PVP: poly(vinyl pyrrolidone); PEI: Polyethylenimine; Fc: Ferrocene; GDH: Glucose dehydrogenase; NQ-LPEI: napthoquinone-4-glycidyl-modified linear poly(ethylenimine); AnMWCNT: anthracene multi walled carbon nanotubes.

None of the references cited in Table 2 include stability data after 30 or 50 days. Ref 6 uses complex electrode architectures, carbon based materials, and enzyme immobilization protocols. Ref 7 uses carbon based materials, complex architecture and dual enzyme at the anode (they use catalase to transform the hydrogen peroxide that is formed). Ref 25 uses complicated fabrication protocols and involves again carbon materials and enzyme immobilization. Of note as well that all these references (cited in Table 2) use an oxygen reductase enzyme at the cathode, to bolster the catalytic the reaction at the cathode and the EFC performance. The advantages of the disclosed biofuel cell is its simplicity (with respect to fabrication) and at least the fact that despite not having an enzyme at the cathode, the disclosed device is still able to produce enough power for real applications.

### Example 8. The EFC is a Self-Powered Glucose Sensor

Materials and Methods

For the experiments using saliva, the saliva of healthy volunteers was collected after fasting (12 hours). The glucose concentration in these samples was determined through the use of a commercial Glucose Assay Kit (GAGO-20, Sigma Aldrich) and a spectrophotometer (Promega). To mimic physiological variations of glucose in saliva, different concentrations of glucose were added to this sample. All protocols and procedures involving human saliva were approved by the KAUST Institutional Biosafety and Bioethics Committee (IBEC). The volunteers provided signed consent to participate in the study. Saliva samples were collected and frozen at −20° C. Fresh solutions were made for each new measurement.

Results

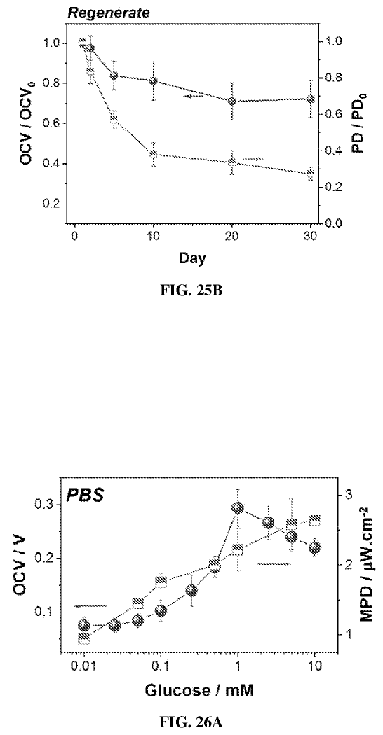

Since glucose is oxidized at the anode by GOx, the current generated by this reaction is proportional to analyte concentration along with the power output of the biofuel cell. Therefore, the change in the OCV and MPD of the EFC as a function of glucose concentration in saliva and PBS (concentration range in saliva: 10 μM-1 mM, in PBS: 1 μM-10 mM, FIGS. 26A and 26B) was measured. Both the OCV and the MPD generated by the biofuel cell increase with glucose concentration, demonstrating the use of the EFC as a self-powered glucose sensor in physiological fluids. At higher glucose concentrations, OCV decreases because of mass transport limitations. Membrane-free devices follow a similar trend, validating the glucose activated power generation of our EFCs (FIG. 20B).

In additional studies, three EFCs were connected in series polarizing a capacitor (100 μF) to draw power in PBS.

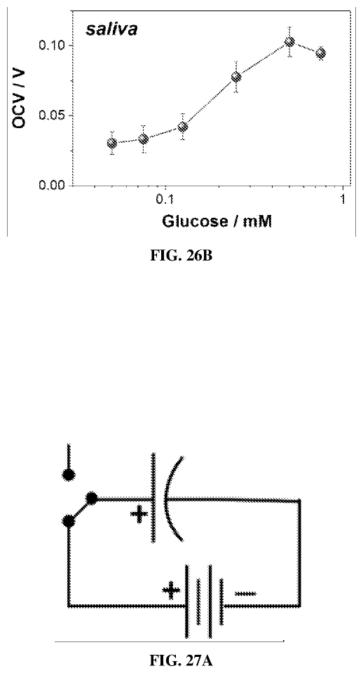

A total output of 1.1 V in PBS was drawn from the three EFCs connected in series (FIGS. 27A and 27B). When fueled by 1 mM glucose in PBS, this EFC platform was able to drive an LED (data not shown).

The membrane-free EFC configuration also effectively powered the device as the bioanode and cathode were immersed in PBS containing glucose.

### Example 9. The EFC Generates Sufficient Power to Drive Organic Electrochemical Transistor (OECT)

Materials and Methods

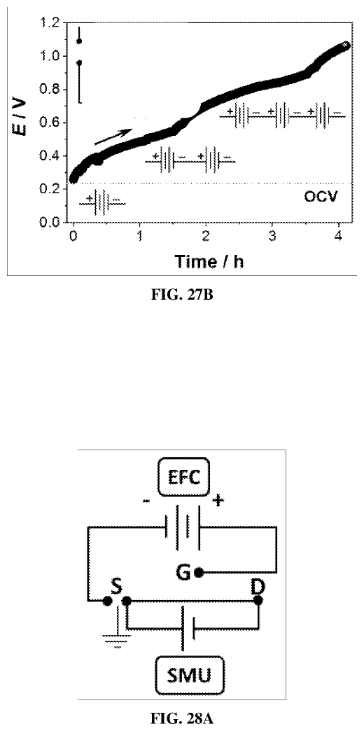

The OECT biosensor is depicted in FIG. 28A. The channel of this OECT is made of PEDOT:PSS. The dimension of the channels was 10 μm in length and 100 μm in width, whereas the Au electrode used as the gate had an area of 500×500 μm².

Results

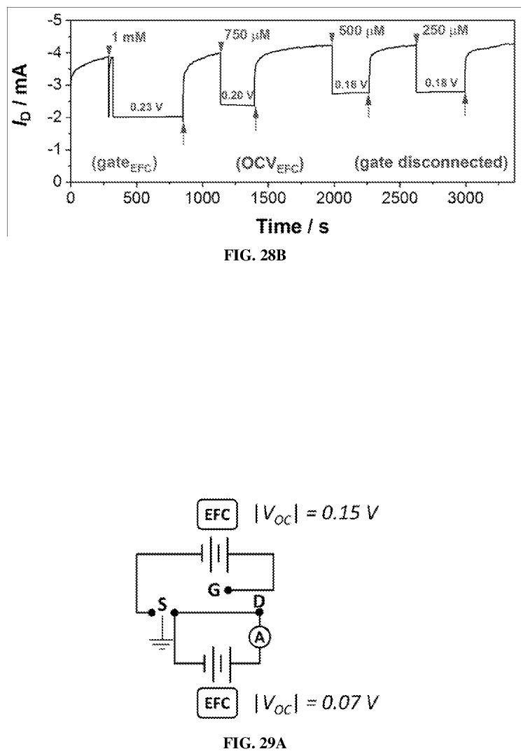

A membrane-free EFC can switch ON and OFF an OECT in a fully-integrated platform (FIG. 28A). The OECT operates in depletion mode, meaning that the drain current decreases upon application of a gate voltage. As the EFC provides the power to apply a voltage at the gate electrode, the drain current decreases, consistent with the OECT operation. Upon supplying more glucose to the EFC, the OECT current decreases further (note that the glucose content was decreased to show that the device performance is not dependent on a stepwise increase in biofuel concentration) (FIG. 28B). The OCV depends on glucose concentration to control the gate voltage. The power of EFCs can be used to bias both the gate and the channel simultaneously. FIGS. 29A and 29B demonstrates the real-time changes in the source-drain current of a fully EFC powered accumulation mode OECT (based on a p-type organic semiconducting channel) as the polarity of the EFC biasing the gate electrode is reversed (Savva, et al., Chemistry of Materials 2019, 31 (3), 927-937). This configuration demonstrates the strategy to fabricate low-cost, stable, polymeric biofuel cells that utilize glucose to power other electronic devices.

The enzymatic biofuel cell, in its simplest design, exhibits superior performances (Table 1) compared to previously described biofuel cells at physiologically relevant glucose concentration in that: (i) it avoids the tedious processes of enzyme immobilization, (ii) it does not need an electron mediator, (iii) it is simple and scalable, (iv) it can be utilized as a self-powered multi-analyte sensors, (v) the enzymatic biofuel cell is stable for at least a month, and in some embodiments, for more than a month (vi) it does not need oxygen reductase at the cathode, (vii) it performs at ambient oxygen concentration and physiological pH, and (viii) the electrodes are made of conducting polymers coated on a thin layer of metal, which reduces the cost related to conventional electrodes made of thick and expensive catalytically active metals.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

### REFERENCES

1. Pappa, A.-M.; Parlak, O.; Scheiblin, G.; Mailley, P.; Salleo, A.; Owens, R. M., Organic Electronics for Point-of-Care Metabolite Monitoring. Trends in Biotechnology 2018, 36 (1), 45-59.
2. Gifford, R., Continuous Glucose Monitoring: 40 Years, What We've Learned and What's Next. ChemPhysChem 2013, 14 (10), 2032-2044.
3. Witkowska Nery, E.; Kundys, M.; Jeleń, P. S.; Jönsson-Niedziółka, M., Electrochemical Glucose Sensing: Is There Still Room for Improvement? Analytical Chemistry 2016, 88 (23), 11271-11282.
4. Wang, J., Electrochemical Glucose Biosensors. Chemical Reviews 2008, 108 (2), 814-825.
5. Heller, A.; Feldman, B., Electrochemical Glucose Sensors and Their Applications in Diabetes Management. Chemical Reviews 2008, 108 (7), 2482-2505.
6. Freire, R. S.; Pessoa, C. A.; Mello, L. D.; Kubota, L. T., Direct electron transfer: an approach for electrochemical biosensors with higher selectivity and sensitivity. Journal of the Brazilian Chemical Society 2003, 14, 230-243.
7. Wu, Y.; Hu, S., Biosensors based on direct electron transfer in redox proteins. Microchimica Acta 2007, 159 (1), 1-17.
8. Zhang, W.; Li, G., Third-Generation Biosensors Based on the Direct Electron Transfer of Proteins. Analytical Sciences 2004, 20 (4), 603-609.
9. Zhu, C.; Yang, G.; Li, H.; Du, D.; Lin, Y., Electrochemical Sensors and Biosensors Based on Nanomaterials and Nanostructures. Analytical Chemistry 2015, 87 (1), 230-249.
10. Kissinger, P. T., Engineering the Bioelectronic Interface: Applications to Analyte Biosensing and Protein Detection. Journal of the American Chemical Society 2010, 132 (4), 1444-1444.
11. Banica, F. G., Chemical Sensors and Biosensors: Fundamentals and Applications. Wiley: 2012.
12. Simon, D. T.; Gabrielsson, E. O.; Tybrandt, K.; Berggren, M., Organic Bioelectronics: Bridging the Signaling Gap between Biology and Technology. Chemical Reviews 2016, 116 (21), 13009-13041.
13. Slaughter, G.; Kulkarni, T., Enzymatic glucose biofuel cell and its application. Journal of Biochips & Tissue Chips 2015, 5 (1), 1.
14. Minteer, S. D.; Atanassov, P.; Luckarift, H. R.; Johnson, G. R., New materials for biological fuel cells. Materials Today 2012, 15 (4), 166-173.
15. Aghahosseini, H.; Ramazani, A.; Azimzadeh Asiabi, P.; Gouranlou, F.; Hosseini, F.; Rezaei, A.; Min, B.-K.; Woo Joo, S., Glucose-based Biofuel Cells: Nanotechnology as a Vital Science in Biofuel Cells Performance. Nanochemistry Research 2016, 1 (2), 183-204.
16. Kwon, C. H.; Ko, Y.; Shin, D.; Kwon, M.; Park, J.; Bae, W. K.; Lee, S. W.; Cho, J., High-power hybrid biofuel cells using layer-by-layer assembled glucose oxidase-coated metallic cotton fibers. Nature Communications 2018, 9 (1), 4479.
17. Wen, D.; Eychmüller, A., Enzymatic Biofuel Cells on Porous Nanostructures. Small 2016, 12 (34), 4649-4661.
18. Giovannitti, A.; Maria, I. P.; Hanifi, D.; Donahue, M. J.; Bryant, D.; Barth, K. J.; Makdah, B. E.; Savva, A.; Moia, D.; Zetek, M.; Barnes, P. R. F.; Reid, O. G.; Inal, S.; Rumbles, G.; Malliaras, G. G.; Nelson, J.; Rivnay, J.; McCulloch, I., The Role of the Side Chain on the Performance of N-type Conjugated Polymers in Aqueous Electrolytes. Chemistry of Materials 2018, 30 (9), 2945-2953.
19. Liu, J.; Qiu, L.; Alessandri, R.; Qiu, X.; Portale, G.; Dong, J.; Talsma, W.; Ye, G.; Sengrian, A. A.; Souza, P. C. T.; Loi, M. A.; Chiechi, R. C.; Marrink, S. J.; Hummelen, J. C.; Koster, L. J. A., Enhancing Molecular n-Type Doping of Donor-Acceptor Copolymers by Tailoring Side Chains. Advanced Materials 2018, 30 (7), 1704630.
20. Wustoni, S.; Savva, A.; Sun, R.; Bihar, E.; Inal, S., Enzyme-Free Detection of Glucose with a Hybrid Conductive Gel Electrode. Advanced Materials Interfaces 0 (0), 1800928.
21. Savva, A.; Wustoni, S.; Inal, S., Ionic-to-electronic coupling efficiency in PEDOT:PSS films operated in aqueous electrolytes. Journal of Materials Chemistry C 2018.
22. Bihar, E.; Wustoni, S.; Pappa, A. M.; Salama, K. N.; Baran, D.; Inal, S., A fully inkjet-printed disposable glucose sensor on paper. npj Flexible Electronics 2018, 2 (1), 30.
23. Inal, S.; Rivnay, J.; Suiu, A.-O.; Malliaras, G. G.; McCulloch, I., Conjugated Polymers in Bioelectronics. Accounts of Chemical Research 2018, 51 (6), 1368-1376.
24. Willner, I.; Katz, E., Integration of Layered Redox Proteins and Conductive Supports for Bioelectronic Applications. Angewandte Chemie International Edition 2000, 39 (7), 1180-1218.
25. Spyropoulos, G. D.; Gelinas, J. N.; Khodagholy, D., Internal ion-gated organic electrochemical transistor: A building block for integrated bioelectronics. Science Advances 2019, 5 (2), eaau7378.
26. Pappa, A. M.; Ohayon, D.; Giovannitti, A.; Maria, I. P.; Savva, A.; Uguz, I.; Rivnay, J.; McCulloch, I.; Owens, R. M.; Inal, S., Direct metabolite detection with an n-type accumulation mode organic electrochemical transistor. Science Advances 2018, 4 (6).
27. Liao, C.; Zhang, M.; Niu, L.; Zheng, Z.; Yan, F., Highly selective and sensitive glucose sensors based on organic electrochemical transistors with graphene-modified gate electrodes. Journal of Materials Chemistry B 2013, 1 (31), 3820-3829.
28. Piro, B.; Mattana, G.; Zrig, S.; Anquetin, G.; Battaglini, N.; Capitao, D.; Maurin, A.; Reisberg, S., Fabrication and Use of Organic Electrochemical Transistors for Sensing of Metabolites in Aqueous Media. Applied Sciences 2018, 8 (6), 928.
29. Lineweaver, H.; Burk, D., The Determination of Enzyme Dissociation Constants. Journal of the American Chemical Society 1934, 56 (3), 658-666.
30. Milton Ross, D.; Minteer Shelley, D., Direct enzymatic bioelectrocatalysis: differentiating between myth and reality. Journal of The Royal Society Interface 2017, 14 (131), 20170253.
31. Trefz, D.; Ruff, A.; Tkachov, R.; Wieland, M.; Goll, M.; Kiriy, A.; Ludwigs, S., Electrochemical Investigations of the N-Type Semiconducting Polymer P(NDI2OD-T2) and Its Monomer: New Insights in the Reduction Behavior. The Journal of Physical Chemistry C 2015, 119 (40), 22760-22771.
32. Wooten, M.; Karra, S.; Zhang, M.; Gorski, W., On the Direct Electron Transfer, Sensing, and Enzyme Activity in the Glucose Oxidase/Carbon Nanotubes System. Analytical Chemistry 2014, 86 (1), 752-757.
33. Thompson, B. C.; Winther-Jensen, O.; Vongsvivut, J.; Winther-Jensen, B.; MacFarlane, D. R., Conducting Polymer Enzyme Alloys: Electromaterials Exhibiting Direct Electron Transfer. Macromolecular Rapid Communications 2010, 31 (14), 1293-1297.
34. Zhang, Y.; Savva, A.; Wustoni, S.; Hama, A.; Maria, I. P.; Giovannitti, A.; McCulloch, I.; Inal, S., Visualizing the Solid-Liquid Interface of Conjugated Copolymer Films Using Fluorescent Liposomes. ACS Applied Bio Materials 2018, 1 (5), 1348-1354.
35. Giovannitti, A.; Nielsen, C. B.; Sbircea, D.-T.; Inal, S.; Donahue, M.; Niazi, M. R.; Hanifi, D. A.; Amassian, A.; Malliaras, G. G.; Rivnay, J.; McCulloch, I., N-type organic electrochemical transistors with stability in water. Nature Communications 2016, 7, 13066.
36. Giussani, E.; Fazzi, D.; Brambilla, L.; Caironi, M.; Castiglioni, C., Molecular Level Investigation of the Film Structure of a High Electron Mobility Copolymer via Vibrational Spectroscopy. Macromolecules 2013, 46 (7), 2658-2670.
37. Navarrete, J. T. L.; Zerbi, G., Lattice dynamics and vibrational spectra of polythiophene. II. Effective coordinate theory, doping induced, and photoexcited spectra. The Journal of Chemical Physics 1991, 94 (2), 965-970.
38. Kergoat, L.; Piro, B.; Simon, D. T.; Pham, M.-C.; Noel, V.; Berggren, M., Detection of Glutamate and Acetylcholine with Organic Electrochemical Transistors Based on Conducting Polymer/Platinum Nanoparticle Composites. Advanced Materials 2014, 26 (32), 5658-5664.
39. Pappa, A.-M.; Curto, V. F.; Braendlein, M.; Strakosas, X.; Donahue, M. J.; Fiocchi, M.; Malliaras, G. G.; Owens, R. M., Organic Transistor Arrays Integrated with Finger-Powered Microfluidics for Multianalyte Saliva Testing. Advanced Healthcare Materials 2016, 5 (17), 2295-2302.
40. Braendlein, M.; Pappa, A.-M.; Ferro, M.; Lopresti, A.; Acquaviva, C.; Mamessier, E.; Malliaras, G. G.; Owens, R. M., Lactate Detection in Tumor Cell Cultures Using Organic Transistor Circuits. Advanced Materials 2017, 29 (13), 1605744.
41. Luz, R. A. S.; Pereira, A. R.; de Souza, J. C. P.; Sales, F. C. P. F.; Crespilho, F. N., Enzyme Biofuel Cells: Thermodynamics, Kinetics and Challenges in Applicability. ChemElectroChem 2014, 1 (11), 1751-1777.
42. Mitraka, E.; Jafari, M. J.; Vagin, M.; Liu, X.; Fahlman, M.; Ederth, T.; Berggren, M.; Jonsson, M. P.; Crispin, X., Oxygen-induced doping on reduced PEDOT. Journal of Materials Chemistry A 2017, 5 (9), 4404-4412.
43. Singh, S. K.; Crispin, X.; Zozoulenko, I. V., Oxygen Reduction Reaction in Conducting Polymer PEDOT: Density Functional Theory Study. The Journal of Physical Chemistry C 2017, 121 (22), 12270-12277.
44. Winther-Jensen, B.; Winther-Jensen, 0.; Forsyth, M.; MacFarlane, D. R., High Rates of Oxygen Reduction over a Vapor Phase-Polymerized PEDOT Electrode. Science 2008, 321 (5889), 671-674.
45. Mitraka, E.; Gryszel, M.; Vagin, M.; Jafari, M. J.; Singh, A.; Warczak, M.; Mitrakas, M.; Berggren, M.; Ederth, T.; Zozoulenko, I.; Crispin, X.; Głowacki, E. D., Electrocatalytic Production of Hydrogen Peroxide with Poly(3,4-ethylenedioxythiophene) Electrodes. Advanced Sustainable Systems 2019, 3 (2), 1800110.
46. Eckermann, A. L.; Feld, D. J.; Shaw, J. A.; Meade, T. J., Electrochemistry of redox-active self-assembled monolayers. Coordination Chemistry Reviews 2010, 254 (15), 1769-1802.
47. Salimi, A.; Sharifi, E.; Noorbakhsh, A.; Soltanian, S, Immobilization of glucose oxidase on electrodeposited nickel oxide nanoparticles: Direct electron transfer and electrocatalytic activity. Biosensors and Bioelectronics 2007, 22 (12), 3146-3153.
48. Laviron, E., General expression of the linear potential sweep voltammogram in the case of diffusionless electrochemical systems. Journal of Electroanalytical Chemistry and Interfacial Electrochemistry 1979, 101 (1), 19-28.
49. Wang, S.; Sun, H.; Erdmann, T.; Wang, G.; Fazzi, D.; Lappan, U.; Puttisong, Y.; Chen, Z.; Berggren, M.; Crispin, X.; Kiriy, A.; Voit, B.; Marks, T. J.; Fabiano, S.; Facchetti, A., A Chemically Doped Naphthalenediimide-Bithiazole Polymer for n-Type Organic Thermoelectrics. Advanced Materials 2018, 30 (31), 1801898.
50. Cho, C.; Stevens, B.; Hsu, J.-H.; Bureau, R.; Hagen, D. A.; Regev, O.; Yu, C.; Grunlan, J. C., Completely Organic Multilayer Thin Film with Thermoelectric Power Factor Rivaling Inorganic Tellurides. Advanced Materials 2015, 27 (19), 2996-3001.
51. Rivnay, J.; Toney, M. F.; Zheng, Y.; Kauvar, I. V.; Chen, Z.; Wagner, V.; Facchetti, A.; Salleo, A., Unconventional Face-On Texture and Exceptional In-Plane Order of a High Mobility n-Type Polymer. Advanced Materials 2010, 22 (39), 4359-4363.
52. Miglbauer, E.; Wójcik, P. J.; Glowacki, E. D., Single-compartment hydrogen peroxide fuel cells with poly(3,4-ethylenedioxythiophene) cathodes. Chemical Communications 2018, 54 (84), 11873-11876.
53. Savva, A.; Cendra, C.; Giugni, A.; Tone, B.; Surgailis, J.; Ohayon, D.; Giovannitti, A.; McCulloch, I.; Di Fabrizio, E.; Salleo, A.; Rivnay, J.; Inal, S., Influence of Water on the Performance of Organic Electrochemical Transistors. Chemistry of Materials 2019, 31 (3), 927-937.

1. A biofuel cell comprising:
a bioanode which comprises (a) a conductive substrate; (b) one or more n-type polymers; and (c) one or more enzymes;
a biocathode which comprises (a) a conductive substrate; (b) one or more p-type polymers; and
an electrolyte solution which comprises one or more metabolite capable of reacting with the enzyme,
wherein the bioanode is electrically connected to the biocathode, and wherein the electrolyte solution is in electrical communication with the bioanode and the biocathode.

2. The biofuel cell of claim 1, wherein the n-type polymer is P90.

3. The biofuel cell of claim 1, wherein: (a) the bioanode cell further comprises coating; (b) the biocathode further comprises one or more enzymes.

4. (canceled)

5. The biofuel cell of claim 1, wherein the enzyme is selected from the group consisting of glucose oxidase, glucose dehydrogenase, alcohol dehydrogenase, aldehyde dehydrogenase, formate dehydrogenase, formaldehyde dehydrogenase, lactic dehydrogenase, lactose dehydrogenase, lactate oxidase, cholesterol oxidase, tyrosinase, and pyruvate dehydrogenase.

6. The biofuel cell of claim 1, wherein the enzyme is glucose oxidase.

7. The biofuel cell of claim 1, wherein the p-type polymer is a mixture of PEDOT:PEDOT-OH, optionally, wherein the molar ratio of PEDOT to PEDOT-OH is 1.

8. (canceled)

9. The biofuel cell of claim 1, wherein the biocathode is capable of reducing an oxidant in the presence of electrons to form water.

10. The biofuel cell of claim 9, wherein the oxidant is oxygen, and optionally, wherein the oxygen is at ambient concentration.

11. (canceled)

12. The biofuel cell of claim 4, wherein one or more enzymes are oxygen reductase, and optionally, wherein the oxygen reductase is laccase or bilirubin oxidase.

13. (canceled)

14. The biofuel cell of claim 1, wherein the metabolite is selected from the group consisting of glucose, glucose-1, D-glucose, L-glucose, glucose-6-phosphate, ammonia, methanol, ethanol, propanol, isobutanol, butanol and isopropanol, allyl alcohols, aryl alcohols, glycerol, cholesterol, propanediol, mannitol, glucoronate, aldehyde, carbohydrates, lactate, lactate-6-phosphate, D-lactate, L-lactate, fructose, galactose-1, galactose, aldose, sorbose, mannose, glycerate, coenzyme A, acetyl Co-A, malate, isocitrate, formaldehyde, acetaldehyde, acetate, citrate, L-gluconate, beta-hydroxysteroid, alpha-hydroxysteroid, lactaldehyde, testosterone, gluconate, fatty acids, lipids, phosphoglycerate, retinal, estradiol, cyclopentanol, hexadecanol, long-chain alcohols, coniferyl-alcohol, cinnamyl-alcohol, formate, long-chain aldehydes, pyruvate, butanal, acryl-CoA, steroids, amino acids, favin, NADH, NADH2, NADPH, NADPH2, and hydrogen.

15. The biofuel cell of claim 1, wherein the metabolite is glucose, glucose-1, D-glucose, L-glucose, or glucose-6-phosphate.

16. The biofuel cell of claim 1, wherein the metabolite is glucose, and optionally, wherein the glucose is at a physiological relevant concentration.

17. (canceled)

18. The biofuel cell of claim 1, wherein: (a) the electrolyte solution is a buffer, a biological fluid, or a combination thereof; (b) the electrolyte solution is at a pH between 3 and 8.5; and (c) the reaction of the metabolite and the enzyme produces electrons which are transferred to the polymers of the bioanode.

19. (canceled)

20. The biofuel cell of claim 1, wherein the electrolyte solution is at pH 7.4.

21. (canceled)

22. The biofuel cell of claim 1 further comprising a membrane.

23. The biofuel cell of claim 1, wherein the biofuel cell preserves at least 15%, at least 20%, at least 30%, at least 35%, at least 40%, or at least 45% of its original maximum power density (MPD) after at least 30 days, at least 35 days, at least 40 days, at least 45 days, or at least 50 days.

24. The biofuel cell of claim 1, wherein the biofuel cell preserves at least 20%, at least 30%, at least 35%, at least 40%, at least 45%, at least 50%, at least 55%, at least 60%, at least 65%, or at least 70% of its original open circuit potential (OCP) after at least 30 days, at least 35 days, at least 40 days, at least 45 days, or at least 50 days.

25. The biofuel cell of claim 1, wherein: (a) the power output of the biofuel cell is proportional to the metabolite concentration; (b) the biofuel cell is utilized for powering portable devices and wearable electronics; (c) the biofuel cell is utilized for powering implantable devices; (d) the biofuel cell is utilized as an implanted device.

26. (canceled)

27. (canceled)

28. (canceled)

29. The biofuel cell of claim 1, wherein: (a) the biofuel cell is utilized as an energy source to power a sensor for monitoring metabolites, ions, pH, or temperature; or (b) the biofuel cell is utilized as a self-powered multi-analyte sensor.

30. (canceled)

31. (canceled)

32. A method of generating electrical power contains the steps of oxidizing one or more metabolites in an electrolyte solution at a bioanode and reducing an oxidant at a biocathode, wherein (a) the electrolyte solution is in electrical communication with the bioanode and the biocathode; (b) the bioanode contains a conductive substrate, one or more n-type polymers, and one or more enzymes which can react with the metabolites; (c) the biocathode contains a conductive substrate and one or more p-type polymeric materials, and wherein the bioanode is electrically connected to the biocathode.
